(19) United States
(12) Patent Application Publication
(10) Pub. No.: US 2012/0172384 A1
(43) Pub. Date: Jul. 5, 2012

(54) HETEROCYCLYLAMINOPYRIMIDINES AS KINASE INHIBITORS

(76) Inventors: Mihiro Sunose, Cambridge (GB); Jeremy Major, Cambridge (GB); Richard John Harrison, Cambridge (GB); Jay Freeman, Norwich (GB); Adeline Morel, Essex (GB)

(21) Appl. No.: 13/379,028

(22) PCT Filed: Jun. 17, 2010
(86) PCT No.: PCT/EP10/58574

§ 371 Date: Mar. 9, 2012

(30) Foreign Application Priority Data
Jun. 18, 2009 (EP) 09163098.8

(51) Int. Cl. A61K 31/506; A61P 37/08; A61P 29/00; C07D 401/12; A61P 37/00

(52) U.S. Cl. 514/275; 544/324

(57) ABSTRACT

The invention relates to compounds of formula (I)

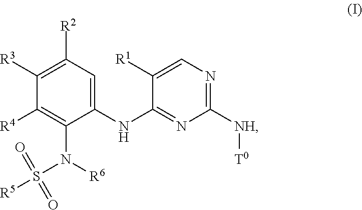

wherein R¹ to R⁶ and T⁰ have the meaning as cited in the description and the claims. Said compounds are useful as inhibitors of ZAP-70, Syk or JAK3 for the treatment or prophylaxis of immunological, inflammatory, autoimmune, allergic disorders, immunologically-mediated diseases. The invention also relates to pharmaceutical compositions including said compounds, the preparation of such compounds as well as the use as medicaments.

The present invention relates to a novel class of kinase inhibitors, including pharmaceutically acceptable salts, prodrugs and metabolites thereof, which are useful for modulating protein kinase activity for modulating cellular activities such as signal transduction, proliferation, and cytokine secretion. More specifically the invention provides compounds which inhibit, regulate and/or modulate kinase activity, in particular ZAP-70, Syk and JAK3 activity, and signal transduction pathways relating to cellular activities as mentioned above. Furthermore, the present invention relates to pharmaceutical compositions comprising said compounds, e.g. for the treatment of diseases such as immunological, inflammatory, autoimmune and allergic disorders, or immunologically-mediated diseases and processes for preparing said compounds.

Protein kinases participate in the signaling events which control the activation, growth and differentiation of cells in response to extracellular mediators or stimuli such as growth factors, cytokines or chemokines. In general, these kinases are classified in two groups, those that preferentially phosphorylate tyrosine residues and those that preferentially phosphorylate serine and/or threonine residues. The tyrosine kinases include membrane-spanning growth factor receptors such as the epidermal growth factor receptor (EGFR) and cytosolic non-receptor kinases such as ZAP-70, Syk or JAK3.

Inappropriately high protein kinase activity is involved in many diseases including inflammatory disorders and cancer. This can be caused either directly or indirectly by the failure of control mechanisms due to mutation, overexpression or inappropriate activation of the enzyme. In all of these instances, selective inhibition of the kinase is expected to have a beneficial effect.

Protein tyrosine kinases—both receptor tyrosine kinases and non-receptor kinases—are essential for the activation and proliferation of cells of the immune system. Among the earliest detectable events upon the immunoreceptor activation in mast cells, T cells and B cells is the stimulation of non-receptor tyrosine kinases. Immune receptors such as the high-affinity IgE receptor (FcεRI), T cell antigen receptor (TCR) and B cell receptor, consist of antigen-binding subunits and signal transducing subunits. The signal transducing chain contains one or more copies of immunoreceptor tyrosine-based activation motifs (ITAMSs).

For TCR activation, ITAMS located in the CD3 molecule are phosphorylated by Lck and Fyn, two Src family tyrosine kinases, followed by recruitment and activation of ZAP-70, a member of the Syk family of tyrosine kinases. These activated tyrosine kinases then phosphorylate downstream adaptor molecules such as LAT (linker for activation of T cells) and SLP-76 (SH2 domain-containing leukocyte protein of 76 kDa). This step leads to the activation of multiple downstream signaling molecules such as inducible T cell kinase (ITK), PLCγ1 and PI3 kinase. For B cell receptor activation, ITAMS are phosphorylated on tyrosine residues by Src-family kinases such as Lyn, Fyn or Blk, followed by Syk recruitment and activation. Tyrosine phosphorylation of BLNK (alos known as SLP-65) and linker for activation of B cells (LAB) by Syk leads to activation of Bruton's tyrosine kinase (Btk), PLCγ2 and PI3K for downstream signalling. The FcεRI receptor is a tetrameric protein consisting of the IgE-binding a chain, a signal-amplifying 13-chain and two disulfide linked γ chains which contain the ITAMS for signal transduction. Cross-linking of the FcεRI immune receptor by multivalent antigens activates the Lyn kinase followed by Src recruitment and activation. The following tyrosine phosphorylation of LAT and SLP-76 and activation of Btk, PLCγ and PI3K leads to mast cell degranulation and activation (Wong, 2005, Current Opinion in Pharmacology 5, 264-271).

ZAP-70 (zeta chain-associated protein of 70 kDa) belongs to the Syk family of tyrosine kinases and is associated with the zeta subunit of the T cell receptor (Au-Yeung et al., 2009. Immunol. Rev. 228(1):41-57). ZAP-70 is primarily expressed in T cells and Natural Killer (NK) cells and plays an essential role in signaling through the TCR. The TCR-mediated activation of T cells is crucial for the immune response. Failure to adequately regulate T cell activation can lead to allergic and autoimmune diseases. Therefore ZAP-70 is considered as an attractive target for the development of immunosuppresive agents for T cell mediated diseases.

Several reports provided genetic evidence that ZAP-70 plays an important role in T cell activation. Mutations in ZAP-70 have been shown to be responsible for an autosomal recessive form of severe combined immunodeficiency syndrome (SCID) in humans (Au-Yeung et al., 2009. Immunol. Rev. 228(1):41-57). This SCID syndrome is characterized by the absence of peripheral CD8+ T cells and by the presence of circulating CD4+ T cells that do not respond to TCR-mediated stimuli in vitro. Targeted disruption of the ZAP-70 gene in mice leads to defects in thymic development and T cell activation. Inhibitors of ZAP-70 may therefore represent drugs useful for the treatment of diseases of the immune system (for example autoimmune diseases) or immunologically-mediated diseases (for example allograft transplant rejection and graft-versus-host disease).

Syk (spleen tyrosine kinase) is a cytoplasmic protein tyrosine kinase which functions as a key mediator of immune receptor signalling in a variety of inflammatory cells including B-cells, macrophages and neutrophils. Syk initiates intracellular signalling once the receptor is engaged by its ligand. Blocking Syk is expected to be beneficial by interrupting the abnormal immune response in both autoimmune and allergic diseases (Kyttaris et al., 2007. Clin. Immunol. 124(3):235-237). It was demonstrated that R406, a small molecule Syk kinase inhibitor, suppresses inflammation and bone erosion in an animal model of rheumatoid arthritis (Pine et al., 2007. Clin. Immunol. 124(3):244-257). In addition, R788, a prodrug of R406, demonstrated efficacy in a clinical study for rheumatoid arthritis (Weinblatt et al., 2008. Arthritis Rheum. 58(11):3309-3318).

Cytokine receptors are transmembrane receptors and can be classified in to five families: gp130 family, IL-2 receptor (γ chain family, growth hormone (single-chain) family, IFN family and gp140 family. Receptors for IL-4 and IL-13 belong to the γ chain family which uses a common γ subunit for signal transduction. Cytokines induce receptor oligomerization and activation of a family of non-receptor tyrosine kinases, the Janus kinases (JAK1, JAK2, JAK3 and TYK2). Each JAK kinase is specifically linked to the cytoplasmic tails of different cytokine receptors, leading to the phosphorylation of the receptors and subsequent recruitment and activation of a family of transcription factors named STAT (signal transducer and activator of transcription) (Wong, 2005, Current Opinion in Pharmacology 5, 264-271).

In mammals, JAK1, JAK2 and TYK2 are ubiquitously expressed. By contrast, the expression of JAK3 is predominantly in hematopoietic cells and it is highly regulated with cell development and activation. This suggests a JAK3 inhibitor as an immunosuppressant with restricted effects in vivo and therefore promising drug for immunosuppression and the treatment of inflammatory diseases such as rheumatoid arthritis (Papageorgiou and Wikman 2004, Trends in Pharmacological Sciences 25(11):558-62). Diseases and disorders associated with JAK3 inhibition are further described, for example in WO-A 01/42246 and WO-A 2008/060301.

Several JAK3 inhibitors have been reported in the literature which may be useful in the medical field (O'Shea et al., 2004. Nat. Rev. Drug Discov. 3(7):555-64). A potent JAK3 inhibitor (CP-690,550) was reported to show efficacy in an animal model of organ transplantation (Changelian et al., 2003, Science 302(5646):875-888) and clinical trials (reviewed in: Pesu et al., 2008. Immunol. Rev. 223, 132-142). The CP-690,550 inhibitor is not selective for the JAK3 kinase and inhibits JAK2 kinase with almost equipotency (Jiang et al., 2008, J. Med. Chem. 51(24):8012-8018). It is expected that a selective JAK3 inhibitor that inhibits JAK3 with greater potency than JAK2 may have advantageous therapeutic properties, because inhibition of JAK2 can cause anemia (Ghoreschi et al., 2009. Nature Immunol. 4, 356-360).

In view of the above, there is a need for providing effective ZAP-70, Syk and JAK3 inhibitors.

Inhibitors of FAK and/or ALK and/or ZAP-70 and/or IGF-IR are described in WO-A 2005/016894. Pyrimidine derivatives exhibiting JAK-3 and JAK-2 kinase inhibiting activities are described in WO-A 2008/009458. Pyrimidine compounds in the treatment of conditions in which modulation of the JAK pathway or inhibition of JAK kinases, particularly JAK3 are described in WO-A 2008/118822 and WO-A 2008/118823.

Thus, an object of the present invention is to provide a new class of compounds as kinase inhibitors, especially as ZAP-70, Syk and JAK3 inhibitors, which may be effective in the treatment or prophylaxis of immunological, inflammatory, autoimmune, allergic disorders, immunologically-mediated diseases or other diseases or disorders associated with ZAP-70, Syk or JAK3.

Accordingly, the present invention provides compounds of formula (I)

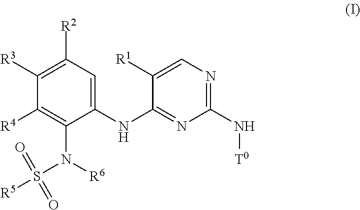

or a pharmaceutically acceptable salt, prodrug or metabolite thereof, wherein
T⁰ is 5 membered aromatic heterocyclyl, wherein T⁰ is optionally substituted with one or more R⁷, which are the same or different;
R⁷ is halogen; CN; C(O)OR⁸; OR⁸; C(O)R⁸; C(O)N(R⁸R^{8a}); S(O)₂N(R⁸R^{8a}); S(O)N(R⁸R^{8a}); S(O)₂R⁸; S(O)R⁸; N(R⁸)S(O)₂N(R^{8a}R^{8b}); SR⁸; N(R⁸R^{8a}); NO₂; OC(O)R⁸; N(R⁸)C(O)R^{8a}; N(R⁸)S(O)₂R^{8a}; N(R⁸)S(O)R^{8a}; N(R⁸)C(O)N(R^{8a}R^{8b}); N(R⁸)C(O)OR^{8a}; OC(O)N(R⁸R^{8a}); C₁₋₆ alkyl; C₂₋₆ alkenyl; C₂₋₆ alkynyl; or T¹, wherein C₁₋₆ alkyl; C₂₋₆ alkenyl; and C₂₋₆ alkynyl are optionally substituted with one or more R⁹, which are the same or different;
R⁸, R^{8a}, R^{8b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁰, which are the same or different;
R⁹, R¹⁰ are independently selected from the group consisting of T¹; halogen; CN; C(O)OR¹¹; OR¹¹; C(O)R¹¹; C(O)N(R¹¹R^{11a}); S(O)₂N(R¹¹R^{11a}); S(O)N(R¹¹R^{11a}); S(O)₂R¹¹; S(O)R¹¹; N(R¹¹)S(O)₂N(R^{11a}R^{11b}); N(R¹¹)S(O)N(R^{11a}R^{11b}); SR¹¹; N(R¹¹R^{11a}) NO₂; OC(O)R¹¹; N(R¹¹)C(O)R^{11a}; N(R¹¹)S(O)₂R^{11a}; N(R¹¹)S(O)R^{11a}; N(R¹¹)C(O)N(R^{11a}R^{11b}); N(R¹¹)C(O)OR^{11a}; and OC(O)N(R¹¹R^{11a});
R¹¹; R^{11a}; R^{11b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T¹ is phenyl; C₃₋₇ cycloalkyl; or 4 to 7 membered heterocyclyl, wherein T¹ is optionally substituted with one or more R¹², which are the same or different;
R¹² is halogen; CN; C(O)OR¹³; OR¹³; oxo (═O), where the ring is at least partially saturated; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); N(R¹³)S(O)N(R^{13a}R^{13b}); SR¹³; N(R¹³R^{13a}); NO₂; OC(O)R¹³; N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R¹⁴, which are the same or different;
R¹³, R^{13a}, R^{13b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁵, which are the same or different;
R¹⁴, R¹⁵ are independently selected from the group consisting of halogen; CN; C(O)OR¹⁶; OR¹⁶; C(O)R¹⁶; C(O)N(R¹⁶R^{16a}); S(O)₂N(R¹⁶R^{16a}); S(O)N(R¹⁶R^{16a}); S(O)₂R¹⁶; S(O)R¹⁶; N(R¹⁶)S(O)₂N(R^{16a}R^{16b}); N(R¹⁶)S(O)N(R^{16a}R^{16b}); SR¹⁶; N(R¹⁶R^{16a}); NO₂; OC(O)R¹⁶; N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; and OC(O)N(R¹⁶R^{16a});
R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R¹ is H; F; Cl; Br; CN; C₁₋₄ alkyl; CH₂F; CHF₂; CF₃; OH; OCH₃; NO₂; NH₂; NHCH₃; N(CH₃)₂; or NO₂;
R², R³, R⁴ are independently selected from the group consisting of H; halogen; CN; C(O)OR¹⁷; OR¹⁷; C(O)R¹⁷; C(O)N(R¹⁷R^{17a}); S(O)₂N(R¹⁷R^{17a}); S(O)N(R¹⁷R^{17a}); S(O)₂R¹⁷; S(O)R¹⁷; SR¹⁷; N(R¹⁷R^{17a}); NO₂; OC(O)R¹⁷; N(R¹⁷)C(O)R^{17a}; N(R¹⁷)S(O)₂R^{17a}; N(R¹⁷)S(O)R^{17a}; N(R¹⁷)C(O)N(R^{17a}R¹⁷); N(R¹⁷)C(O)OR^{17a}; OC(O)N(R¹⁷R^{17a}); C₁₋₆ alkyl; C₂₋₆ alkenyl; C₂₋₆ alkynyl; and T², wherein C₁₋₆ alkyl; C₂₋₆ alkenyl; and C₂₋₆ alkynyl are optionally substituted with one or more R¹⁸, which are the same or different;
Optionally, one of the pairs R²/R³, R³/R⁴ is joined together with the phenyl ring to which it is attached to form a bicyclic ring T³;
R¹⁷, R^{17a}, R^{17b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁹, which are the same or different;
R¹⁸, R¹⁹ are independently selected from the group consisting of T²; halogen; CN; C(O)OR²⁰; OR²⁰; C(O)R²⁰; C(O)N(R²⁰R^{20a}); S(O)₂N(R²⁰R^{20a}); S(O)N(R²⁰R^{20a}); S(O)₂R²⁰; S(O)R²⁰) N(R²⁰)S(O)₂N(R²⁰R^{20b}); N(R²⁰)S(O)N(R^{20a}R^{20b}); SR²⁰; N(R²⁰R^{20a}); NO₂; OC(O)R²⁰); N(R²⁰)C(O)R^{20a}; N(R²⁰)S(O)₂R^{20a}); N(R²⁰)S(O)R^{20a}); N(R²⁰)C(O)N(R^{20a}R^{20b}); N(R²⁰)C(O)OR^{20a}; and OC(O)N(R²⁰R^{20a});
R²⁰, R^{20a}, R^{20b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T² is phenyl; C₃₋₇ cycloalkyl; or 4 to 7 membered heterocyclyl, wherein T² is optionally substituted with one or more R²¹, which are the same or different;
T³ is naphthyl; indenyl; indanyl; or 9 to 11 membered benzo-fused heterobicyclyl, wherein T³ is optionally substituted with one or more R²², which are the same or different;
R²¹, R²² are independently selected from the group consisting of halogen; CN; C(O)OR²³; OR²³; oxo (═O), where the ring is at least partially saturated; C(O)R²³; C(O)N(R²³R^{23a}); S(O)₂N(R²³R^{23a}); S(O)N(R²³R^{23a}); S(O)₂R²³; S(O)R²³; N(R²³)S(O)₂N(R^{23a}R^{23b}); N(R²³)S(O)N(R^{23a}R^{23b}); SR²³; N(R²³R^{23a}); NO₂; OC(O)R²³; N(R²³)C(O)R^{23a}; N(R²³)S(O)₂R^{23a}; N(R²³)S(O)R^{23a}; N(R²³)C(O)N(R^{23a}R^{23b}); N(R²³)C(O)OR^{23a}; OC(O)N(R²³R^{23a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R²³, R^{23a}; R^{23b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

### R⁵ is R²⁴; or N(R²⁴R^{24a});

R²⁴ is T⁴; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R²⁵, which are the same or different;
R^{24a} is H; or C₁₋₄ alkyl, wherein C₁₋₄ alkyl is optionally substituted with one or more F;
R²⁵ is T⁴; halogen; CN; C(O)OR²⁶; OR²⁶; C(O)R²⁶; C(O)N(R²⁶R^{26a}); S(O)₂N(R²⁶R^{26a}); S(O)N(R²⁶R^{26a}); S(O)₂R²⁶; S(O)R²⁶; N(R²⁶)S(O)₂N(R^{26a}R^{26b}); N(R²⁶)S(O)N(R^{26a}R^{26b}); SR²⁶; N(R²⁶R^{26a}); NO₂; OC(O)R²⁶; N(R²⁶)C(O)R^{26a}; N(R²⁶)S(O)₂R^{26a}; N(R²⁶)S(O)R^{26a}; N(R²⁶)C(O)N(R^{26a}R^{26b}); N(R²⁶)C(O)OR^{26a}; or OC(O)N(R²⁶R^{26a});
R²⁶, R^{26a}, R^{26b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T⁴ is phenyl; C₃₋₇ cycloalkyl; or 4 to 7 membered heterocyclyl, wherein T⁴ is optionally substituted with one or more R²⁷, which are the same or different;
R²⁷ is halogen; CN; C(O)OR²⁸; OR²⁸; oxo (═O), where the ring is at least partially saturated; C(O)R²⁸; C(O)N(R²⁸R^{28a}); S(O)₂N(R²⁸R^{28a}); S(O)N(R²⁸R^{28a}); S(O)₂R²⁸; S(O)R²⁸; N(R²⁸)S(O)₂N(R^{28a}R²⁸); N(R²⁸)S(O)N(R^{28a}R²⁸); SR²⁸; N(R²⁸R^{28a}); NO₂; OC(O)R²⁸; N(R²⁸)C(O)R^{28a}; N(R²⁸)S(O)₂R^{28a}; N(R²⁸)S(O)R^{28a}; N(R²⁸)C(O)N(R^{28a}R^{28b}); N(R²⁸)C(O)OR^{28a}; OC(O)N(R²⁸R^{28a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;
R²⁸; R^{28a}; R^{28b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R⁶ is H; C₁₋₄ alkyl; C₃₋₅ cycloalkyl; or C₃₋₅ cycloalkylmethyl, wherein C₁₋₄ alkyl; C₃₋₅ cycloalkyl and C₃₋₅ cycloalkylmethyl are optionally substituted with one or more halogen, which are the same or different.

In case a variable or substituent can be selected from a group of different variants and such variable or substituent occurs more than once the respective variants can be the same or different.

Within the meaning of the present invention the terms are used as follows:

“Alkyl” means a straight-chain or branched saturated hydrocarbon chain. Each hydrogen of an alkyl carbon may be replaced by a substituent.

“Alkenyl” means a straight-chain or branched hydrocarbon chain, that contains at least one carbon-carbon double bond. Each hydrogen of an alkenyl carbon may be replaced by a substituent.

“Alkynyl” means a straight-chain or branched hydrocarbon chain, that contains at least one carbon-carbon triple bond. Each hydrogen of an alkynyl carbon may be replaced by a substituent.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —C(CH₂)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl, tert-butyl, n-pentyl, n-hexyl, or e.g. —CH₂—, —CH₂—CH₂—, —CH(CH₃)—, —C(CH₂)—, —CH₂—CH₂ CH₂—, —CH(C₂H₅)—, —C(CH₃)₂—, when two moieties of a molecule are linked by the alkyl group. Each hydrogen of a C₁₋₆ alkyl carbon may be replaced by a substituent.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Each hydrogen of a C₂₋₆ alkenyl carbon may be replaced by a substituent.

“C₂₋₆ alkynyl” means an alkynyl chain having 2 to 6 carbon atoms, e.g. if present at the end of a molecule: —C≡CH, —CH₂—C≡CH, CH₂—CH₂—C≡CH, CH₂—C≡C—CH₃, or e.g. —C≡C— when two moieties of a molecule are linked by the alkynyl group. Each hydrogen of a C₂₋₆ alkynyl carbon may be replaced by a substituent.

“C₃₋₇ cycloalkyl” or “C₃₋₇ cycloalkyl ring” means a cyclic alkyl chain having 3-7 carbon atoms, e.g. cyclopropyl, cyclobutyl, cyclopentyl, cyclohexyl, cyclohexenyl, cycloheptyl. Each hydrogen of a cycloalkyl carbon may be replaced by a substituent. Accordingly, “C₃₋₅ cycloalkyl” means a cycloalkyl having 3 to 5 carbon atoms.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine.

“9 to 11 membered heterobicyclyl” or “9 to 11 membered heterobicycle” means a heterocyclic system of two rings with 9 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 9 to 11 membered heterobicycle are indole, indoline, benzofuran, benzothiophene, benzoxazole, benzisoxazole, benzothiazole, benzisothiazole, benzimidazole, benzimidazo line, quinoline, quinazoline, dihydroquinazoline, quinoline, dihydroquinoline, tetrahydroquinoline, decahydroquinoline, isoquino line, decahydroisoquinoline, tetrahydroisoquinoline, dihydroisoquinoline, benzazepine, purine or pteridine. The term 9 to 11 membered heterobicycle also includes spiro structures of two rings like 1,4-dioxa-8-azaspiro[4.5]decane or bridged heterocycles like 8-aza-bicyclo[3.2.1]octane.

“benzofused” heterobicyclyl or “benzofused” heterobicycle means that one of the two rings of the bicycle is a benzene ring.

“5 membered aromatic heterocyclyl” or “5 membered aromatic heterocycle” means a heterocycle derived from cyclopentadienyl or benzene, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, oxadiazole, thiadiazole, triazole, tetrazole.

Preferred compounds of formula (I) are those compounds in which one or more of the residues contained therein have the meanings given below, with all combinations of preferred substituent definitions being a subject of the present invention. With respect to all preferred compounds of the formula (I) the present invention also includes all tautomeric and stereoisomeric forms and mixtures thereof in all ratios, and their pharmaceutically acceptable salts.

In preferred embodiments of the present invention, the substituents mentioned below independently have the following meaning. Hence, one or more of these substituents can have the preferred or more preferred meanings given below.

Preferably, T⁰ is a 5 membered aromatic heterocycle which is attached to the amine in formula (I) via a carbon ring atom and wherein T⁰ is unsubstituted or substituted with one, two or three R⁷, which are the same or different.

Preferably, T⁰ is unsubstituted or substituted and selected from the group consisting of pyrazole; isoxazole; and oxadiazole. More preferably, T⁰ is an unsubstituted or substituted pyrazole.

Preferably, R⁷ is unsubstituted O—C₁₋₆ alkyl; unsubstituted C₁₋₆ alkyl; or T¹.

Preferably, R¹ is H; F; Cl; Br; CH₃; or CF₃. More preferably, R¹ is H; CH₃; Br; Cl; or F. Even more preferably, R¹ is Cl.

Preferably, at least two of R², R³, R⁴ are H.

Preferably, R², R³, R⁴ are independently selected from the group consisting of H; F; Cl; unsubstituted C₁₋₆ alkyl; and unsubstituted O—C₁₋₆ alkyl.

Preferably, R⁵ is R²⁴.

Preferably, R²⁴ is unsubstituted C₁₋₄ alkyl. More preferably, R²⁴ is methyl.

Preferably, R⁶ is H.

Compounds of formula (I) in which some or all of the above-mentioned groups have the preferred meanings are also an object of the present invention.

Further preferred compounds of the present invention are selected from the group consisting of:
N-(2-(5-fluoro-2-(1-methyl-1H-pyrazol-3-ylamino)pyrimidin-4-ylamino)phenyl)methanesulfonamide;
N-(2-(5-chloro-2-(3-methyl-1H-pyrazol-4-ylamino)pyrimidin-4-ylamino)-5-methoxyphenyl)methanesulfonamide;
N-(2-(5-chloro-2-(isoxazol-3-ylamino)pyrimidin-4-ylamino)-5-methoxyphenyl)methanesulfonamide;
N-(2-(5-chloro-2-(1-methyl-1H-pyrazol-3-ylamino)pyrimidin-4-ylamino)-5-methoxyphenyl)methanesulfonamide;
N-(2-(5-chloro-2-(1-methyl-1H-pyrazol-5-ylamino)pyrimidin-4-ylamino)-5-methoxyphenyl)methanesulfonamide;
N-(2-(5-chloro-2-(1-methyl-1H-pyrazol-4-ylamino)pyrimidin-4-ylamino)-5-methoxyphenyl)methanesulfonamide;
N-(2-(5-fluoro-2-(1-methyl-1H-pyrazol-3-ylamino)pyrimidin-4-ylamino)-5-methoxyphenyl)methanesulfonamide;
N-(2-(5-chloro-2-(1,3-dimethyl-1H-pyrazol-5-ylamino)pyrimidin-4-ylamino)-5-methoxyphenyl)methanesulfonamide;
N-(2-(5-chloro-2-(3-cyclopropyl-1-methyl-1H-pyrazol-5-ylamino)pyrimidin-4-ylamino)-5-methoxyphenyl)methanesulfonamide;
N-(2-(2-(3-tert-butyl-1-methyl-1H-pyrazol-5-ylamino)-5-chloropyrimidin-4-ylamino)-5-methoxyphenyl)methanesulfonamide;
N-(2-(5-chloro-2-(3-isopropyl-1-methyl-1H-pyrazol-5-ylamino)pyrimidin-4-ylamino)-5-methoxyphenyl)methanesulfonamide;
N-(2-(5-chloro-2-(1-ethyl-3-methyl-1H-pyrazol-5-ylamino)pyrimidin-4-ylamino)-5-methoxyphenyl)methanesulfonamide;
N-(2-(5-chloro-2-(1,3,5-trimethyl-1H-pyrazol-4-ylamino)pyrimidin-4-ylamino)-5-methoxyphenyl)methanesulfonamide;
N-(2-(5-chloro-2-(1,4-dimethyl-1H-pyrazol-5-ylamino)pyrimidin-4-ylamino)-5-methoxyphenyl)methanesulfonamide;
N-(2-(5-chloro-2-(3-ethyl-1,4-dimethyl-1H-pyrazol-5-ylamino)pyrimidin-4-ylamino)-5-methoxyphenyl)methanesulfonamide;
N-(2-(5-chloro-2-(1-methyl-1H-pyrazol-3-ylamino)pyrimidin-4-ylamino)phenyl)methanesulfonamide;
N-(2-(5-chloro-2-(4-methoxy-1,2,5-oxadiazol-3-ylamino)pyrimidin-4-ylamino)-5-methoxyphenyl)methanesulfonamide;
N-(2-(5-chloro-2-(1,3-dimethyl-1H-pyrazol-4-ylamino)pyrimidin-4-ylamino)-5-methoxyphenyl)methanesulfonamide;
N-(2-(5-chloro-2-(3,5-dimethylisoxazol-4-ylamino)pyrimidin-4-ylamino)-5-methoxyphenyl)methanesulfonamide;
N-(2-(5-chloro-2-(1-methyl-1H-pyrazol-4-ylamino)pyrimidin-4-ylamino)phenyl)methanesulfonamide;
N-(2-(5-fluoro-2-(1-methyl-1H-pyrazol-4-ylamino)pyrimidin-4-ylamino)-5-methoxyphenyl)methanesulfonamide;
N-(2-(5-fluoro-2-(1-methyl-1H-pyrazol-4-ylamino)pyrimidin-4-ylamino)phenyl)methanesulfonamide;
N-(2-(2-(1H-pyrazol-4-ylamino)-5-fluoropyrimidin-4-ylamino)phenyl)methanesulfonamide;
N-(2-(2-(1H-pyrazol-4-ylamino)-5-fluoropyrimidin-4-ylamino)-5-methoxyphenyl)methanesulfonamide;
N-(2-(2-(1H-pyrazol-4-ylamino)-5-chloropyrimidin-4-ylamino)phenyl)methanesulfonamide;
N-(2-(5-chloro-2-(1-ethyl-4-methyl-1H-pyrazol-5-ylamino)pyrimidin-4-ylamino)-5-methoxyphenyl)methanesulfonamide;
N-(2-(2-(1-sec-butyl-3-methyl-1H-pyrazol-5-ylamino)-5-chloropyrimidin-4-ylamino)-5-methoxyphenyl)methanesulfonamide;
N-(2-(5-chloro-2-(1-ethyl-3-methyl-1H-pyrazol-4-ylamino)pyrimidin-4-ylamino)-5-methoxyphenyl)methanesulfonamide;
N-(2-(5-chloro-2-(1-ethyl-1H-pyrazol-5-ylamino)pyrimidin-4-ylamino)-5-methoxyphenyl)methanesulfonamide;
N-(2-(2-(1,3-dimethyl-1H-pyrazol-4-ylamino)-5-fluoropyrimidin-4-ylamino)phenyl)methanesulfonamide;
N-(2-(5-chloro-2-(4-methyl-1,2,5-oxadiazol-3-ylamino)pyrimidin-4-ylamino)-5-methoxyphenyl)methanesulfonamide;
N-(2-(2-(1,3-dimethyl-1H-pyrazol-4-ylamino)-5-fluoropyrimidin-4-ylamino)-5-methoxyphenyl)methanesulfonamide;
N-(2-(5-chloro-2-(1-ethyl-3-methyl-1H-pyrazol-4-ylamino)pyrimidin-4-ylamino)phenyl)methanesulfonamide;
N-(2-(2-(1-ethyl-3-methyl-1H-pyrazol-4-ylamino)-5-fluoropyrimidin-4-ylamino)-5-methoxyphenyl)methanesulfonamide;
N-(2-(5-chloro-2-(1,3-dimethyl-1H-pyrazol-4-ylamino)pyrimidin-4-ylamino)phenyl)methanesulfonamide;
N-(2-(5-fluoro-2-(isoxazol-3-ylamino)pyrimidin-4-ylamino)-5-methoxyphenyl)methanesulfonamide;
N-(2-(2-(1-ethyl-3-methyl-1H-pyrazol-4-ylamino)-5-fluoropyrimidin-4-ylamino)phenyl)methanesulfonamide;
N-(2-(5-chloro-2-(isoxazol-3-ylamino)pyrimidin-4-ylamino)phenyl)methanesulfonamide;
N-(2-(5-chloro-2-(1,5-dimethyl-1H-pyrazol-3-ylamino)pyrimidin-4-ylamino)phenyl)methanesulfonamide;
N-(2-(5-chloro-2-(1-isopropyl-1H-pyrazol-3-ylamino)pyrimidin-4-ylamino)phenyl)methane sulfonamide;
N-(2-(5-bromo-2-(1-methyl-1H-pyrazol-4-ylamino)pyrimidin-4-ylamino)phenyl)methanesulfonamide;
N-(2-(5-chloro-2-(1-ethyl-1H-pyrazol-3-ylamino)pyrimidin-4-ylamino)phenyl)methanesulfonamide;
N-(2-((5-bromo-2-((1-methyl-1H-pyrazol-3-yl)amino)pyrimidin-4-yl)amino)phenyl)methanesulfonamide;
N-(2-((5-chloro-2-(isoxazol-4-ylamino)pyrimidin-4-yl)amino)phenyl)methanesulfonamide;
N-(2-((5-chloro-2-((3-methylisoxazol-5-yl)amino)pyrimidin-4-yl)amino)phenyl)methanesulfonamide;
N-(2-((5-chloro-2-((1-propyl-1H-pyrazol-3-yl)amino)pyrimidin-4-yl)amino)phenyl)methanesulfonamide;
N-(2-((2-((1,5-dimethyl-1H-pyrazol-3-yl)amino)-5-fluoropyrimidin-4-yl)amino)-5-methoxyphenyl)methanesulfonamide;
N-(2-((5-fluoro-2-((1-isopropyl-1H-pyrazol-3-yl)amino)pyrimidin-4-yl)amino)-5-methoxyphenyl)methanesulfonamide;
N-(2-((5-bromo-2-((1-isopropyl-1H-pyrazol-3-yl)amino)pyrimidin-4-yl)amino)phenyl)methanesulfonamide;
N-(2-((5-bromo-2-(isoxazol-3-ylamino)pyrimidin-4-yl)amino)phenyl)methanesulfonamide;
N-(2-((5-bromo-2-((1,5-dimethyl-1H-pyrazol-3-yl)amino)pyrimidin-4-yl)amino)phenyl)methanesulfonamide;
N-(2-((5-chloro-2-((1-(2-methoxyethyl)-1H-pyrazol-4-yl)amino)pyrimidin-4-yl)amino)phenyl)methanesulfonamide
N-(2-((5-chloro-2-((1-methyl-1H-pyrazol-3-yl)amino)pyrimidin-4-yl)amino)phenyl)cyclopropanesulfonamide;
N-(2-((5-chloro-2-((1-isopropyl-1H-pyrazol-3-yl)amino)pyrimidin-4-yl)amino)phenyl)cyclopropanesulfonamide;
N-(2-((5-chloro-2-((1,5-dimethyl-1H-pyrazol-3-yl)amino)pyrimidin-4-yl)amino)phenyl)cyclopropanesulfonamide;
N-(2-((5-chloro-2-((1-methyl-1H-pyrazol-3-yl)amino)pyrimidin-4-yl)amino)phenyl)ethanesulfonamide;
N-(2-((5-chloro-2-((1-methyl-1H-pyrazol-4-yl)amino)pyrimidin-4-yl)amino)phenyl)ethanesulfonamide;
N-(2-((5-chloro-2-((1-methyl-1H-pyrazol-4-yl)amino)pyrimidin-4-yl)amino)phenyl)cyclopropanesulfonamide;
N-(2-((5-chloro-2-((1-ethyl-5-methyl-1H-pyrazol-3-yl)amino)pyrimidin-4-yl)amino)phenyl)methanesulfonamide;
N-(2-((5-chloro-2-((1-(cyclopropylmethyl)-1H-pyrazol-5-yl)amino)pyrimidin-4-yl)amino)-5-methoxyphenyl)methanesulfonamide;
N-(2-((2-((1,5-dimethyl-1H-pyrazol-3-yl)amino)-5-fluoropyrimidin-4-yl)amino)phenyl)ethanesulfonamide;
N-(2-((5-fluoro-2-((1-methyl-1H-pyrazol-4-yl)amino)pyrimidin-4-yl)amino)phenyl)ethanesulfonamide;
N-(2-((5-fluoro-2-((1-methyl-1H-pyrazol-3-yl)amino)pyrimidin-4-yl)amino)phenyl)ethanesulfonamide;
N-(2-((5-fluoro-2-((1-(2-methoxyethyl)-1H-pyrazol-3-yl)amino)pyrimidin-4-yl)amino)phenyl)ethanesulfonamide;
N-(2-((5-chloro-2-((1-(3-cyanopropyl)-1H-pyrazol-4-yl)amino)pyrimidin-4-yl)amino)phenyl)methanesulfonamide;
N-(2-((2-((1-(3-cyanopropyl)-1H-pyrazol-4-yl)amino)-5-fluoropyrimidin-4-yl)amino)phenyl)methanesulfonamide;
N-(2-((5-chloro-2-((1,5-dimethyl-1H-pyrazol-4-yl)amino)pyrimidin-4-yl)amino)-5-methoxyphenyl)methanesulfonamide;
N-(2-((5-chloro-2-((1-isopropyl-1H-pyrazol-5-yl)amino)pyrimidin-4-yl)amino)-5-methoxyphenyl)methanesulfonamide;
N-(2-((5-chloro-2-((3-methyl-1-propyl-1H-pyrazol-5-yl)amino)pyrimidin-4-yl)amino)-5-methoxyphenyl)methanesulfonamide;
N-(2-((5-chloro-2-((1-methyl-1H-pyrazol-3-yl)amino)pyrimidin-4-yl)amino)-6-fluorophenyl)methanesulfonamide;
N-(2-((5-chloro-2-((1-isopropyl-3-methyl-1H-pyrazol-5-yl)amino)pyrimidin-4-yl)amino)-5-methoxyphenyl)methanesulfonamide;
N-(2-((2-((1-(2,2-difluoroethyl)-3-methyl-1H-pyrazol-4-yl)amino)-5-fluoropyrimidin-4-yl)amino)phenyl)methanesulfonamide;
N-(2-((2-((1-(2,2-difluoroethyl)-3-methoxy-1H-pyrazol-4-yl)amino)-5-fluoropyrimidin-4-yl)amino)phenyl)methanesulfonamide;
N-(2-((5-fluoro-2-((3-methoxy-1-(2,2,2-trifluoroethyl)-1H-pyrazol-4-yl)amino)pyrimidin-4-yl)amino)phenyl)methanesulfonamide;
N-(2-((5-fluoro-2-((5-methyl-1-(2,2,2-trifluoroethyl)-1H-pyrazol-3-yl)amino)pyrimidin-4-yl)amino)phenyl)methanesulfonamide;
N-(2-((5-chloro-2-((1-propyl-1H-pyrazol-5-yl)amino)pyrimidin-4-yl)amino)-5-methoxyphenyl)methanesulfonamide;
N-(2-((5-chloro-2-((1-ethyl-5-methyl-1H-pyrazol-4-yl)amino)pyrimidin-4-yl)amino)-5-methoxyphenyl)methanesulfonamide;
N-(2-((5-chloro-2-((1,5-dimethyl-1H-pyrazol-3-yl)amino)pyrimidin-4-yl)amino)-6-fluorophenyl)methanesulfonamide;
N-(2-((5-chloro-2-((1-propyl-1H-pyrazol-3-yl)amino)pyrimidin-4-yl)amino)-6-fluorophenyl)methanesulfonamide;
N-(2-((5-chloro-2-((1-(2,2-difluoroethyl)-3-methoxy-1H-pyrazol-4-yl)amino)pyrimidin-4-yl)amino)phenyl)methanesulfonamide;
N-(2-((5-chloro-2-((1-(2,2-difluoroethyl)-3-methyl-1H-pyrazol-4-yl)amino)pyrimidin-4-yl)amino)phenyl)methanesulfonamide;
N-(2-((5-chloro-2-((3-methoxy-1-(2,2,2-trifluoro ethyl)-1H-pyrazol-4-yl)amino)pyrimidin-4-yl)amino)phenyl)methanesulfonamide;
N-(2-((5-chloro-2-((5-methyl-1-(2,2,2-trifluoroethyl)-1H-pyrazol-3-yl)amino)pyrimidin-4-yl)amino)phenyl)methanesulfonamide;
N-(2-((5-chloro-2-((1-(2,2-difluoroethyl)-1H-pyrazol-4-yl)amino)pyrimidin-4-yl)amino)phenyl)methanesulfonamide;
N-(2-((2-((1-(2,2-difluoroethyl)-1H-pyrazol-4-yl)amino)-5-fluoropyrimidin-4-yl)amino)phenyl)methanesulfonamide;
N-(2-((2-((1-butyl-3-methyl-1H-pyrazol-5-yl)amino)-5-chloropyrimidin-4-yl)amino)-5-methoxyphenyl)methanesulfonamide;
N-(2-((2-((1-butyl-1H-pyrazol-5-yl)amino)-5-chloropyrimidin-4-yl)amino)-5-methoxyphenyl)methanesulfonamide;
N-(2-((5-chloro-2-((1-(1-cyclopropylethyl)-3-methyl-1H-pyrazol-5-yl)amino)pyrimidin-4-yl)amino)-5-methoxyphenyl)methanesulfonamide;
N-(2-((5-chloro-2-((1-methyl-1H-pyrazol-4-yl)amino)pyrimidin-4-yl)amino)-6-fluorophenyl)methanesulfonamide;
N-(2-((5-chloro-2-((1-ethyl-1H-pyrazol-3-yl)amino)pyrimidin-4-yl)amino)-6-fluorophenyl)methanesulfonamide;
N-(2-((5-chloro-2-((1-ethyl-5-methyl-1H-pyrazol-3-yl)amino)pyrimidin-4-yl)amino)-6-fluorophenyl)methanesulfonamide;
3-((5-chloro-4-((2-(methylsulfonamido)phenyl)amino)pyrimidin-2-yl)amino)-1-methyl-1H-pyrazole-5-carboxylic acid;
N-(2-((5-chloro-2-((1-(2-methoxyethyl)-1H-pyrazol-4-yl)amino)pyrimidin-4-yl)amino)-6-fluorophenyl)methanesulfonamide;
N-(2-((5-chloro-2-((1-(2-hydroxyethyl)-3-methyl-1H-pyrazol-5-yl)amino)pyrimidin-4-yl)amino)-5-methoxyphenyl)methanesulfonamide;
N-(2-((5-chloro-2-((1-isobutyl-3-methyl-1H-pyrazol-5-yl)amino)pyrimidin-4-yl)amino)-5-methoxyphenyl)methanesulfonamide;
N-(2-((5-chloro-2-((1-(2-methoxyethyl)-3-methyl-1H-pyrazol-5-yl)amino)pyrimidin-4-yl)amino)-5-methoxyphenyl)methanesulfonamide;
3-((5-chloro-4-((2-(methylsulfonamido)phenyl)amino)pyrimidin-2-yl)amino)-N,1-dimethyl-1H-pyrazole-5-carboxamide
N-(2-((5-chloro-2-((1-isobutyl-1H-pyrazol-5-yl)amino)pyrimidin-4-yl)amino)-5-methoxyphenyl)methanesulfonamide;
N-(2-fluoro-6-((5-fluoro-2-((1-methyl-1H-pyrazol-4-yl)amino)pyrimidin-4-yl)amino)phenyl)methanesulfonamide;
3-((5-chloro-4-((2-(methylsulfonamido)phenyl)amino)pyrimidin-2-yl)amino)-N-ethyl-1-methyl-1H-pyrazole-5-carboxamide; and
3-((5-chloro-4-((2-(methylsulfonamido)phenyl)amino)pyrimidin-2-yl)amino)-N-(2-hydroxyethyl)-1-methyl-1H-pyrazole-5-carboxamide.

Prodrugs of the compounds of the present invention are also within the scope of the present invention.

“Prodrug” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of a prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods.

Metabolites of compounds of formula (I) are also within the scope of the present invention.

The term “metabolites” refers to all molecules derived from any of the compounds according to the present invention in a cell or organism, preferably mammal.

Preferably the term relates to molecules which differ from any molecule which is present in any such cell or organism under physiological conditions

The structure of the metabolites of the compounds according to the present invention will be obvious to any person skilled in the art, using the various appropriate methods.

Where tautomerism, like e.g. keto-enol tautomerism, of compounds of general formula (I) may occur, the individual forms, like e.g. the keto and enol form, are comprised separately and together as mixtures in any ratio. The same applies for stereoisomers, like e.g. enantiomers, cis/trans isomers, conformers and the like.

If desired, isomers can be separated by methods well known in the art, e.g. by liquid chromatography. The same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of formula (I) may be obtained from stereoselective synthesis using optically pure starting materials.

The compounds of formula (I) may exist in crystalline or amorphous form. Furthermore, some of the crystalline forms of the compounds of formula (I) may exist as polymorphs, which are included within the scope of the present invention. Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (ssNMR).

In case the compounds according to formula (I) contain one or more acidic or basic groups, the invention also comprises their corresponding pharmaceutically or toxicologically acceptable salts, in particular their pharmaceutically utilizable salts. Thus, the compounds of the formula (I) which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the formula (I) which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the formula (I) simultaneously contain acidic and basic groups in the molecule, the invention also includes, in addition to the salt forms mentioned, inner salts or betaines (zwitterions). The respective salts according to the formula (I) can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the formula (I) which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means approved by a regulatory agency such as the EMEA (Europe) and/or the FDA (US) and/or any other national regulatory agency for use in animals, preferably in humans.

The present invention furthermore includes all solvates of the compounds according to the invention.

The present invention provides compounds of formula (I) as kinase inhibitors, especially as ZAP-70, Syk or JAK3 inhibitors. The compounds of formula (I) may inhibit the kinase, optionally in addition to other kinases mentioned above without being limited by theory.

Accordingly, the compounds of the present invention are useful for the prevention or treatment of immunological, inflammatory, autoimmune, allergic disorders, or immunologically-mediated diseases, especially acute or chronic inflammation; rheumatoid arthritis; multiple sclerosis; psoriasis; Crohn's disease; ulcerative colitis; systemic lupus erythematosus; asthma; chronic obstructive pulmonary disease (COPD); allergic rhinitis; allograft transplant rejection; graft-versus-host disease; dry eye disorder; or uveitis.

Without intending to be limited by theory, the compounds of the invention are useful for treating or preventing diseases that are mediated directly or indirectly by T cells, B cells, neutrophils or mast cells.

Thus, another aspect of the present invention is a compound of the present invention or a pharmaceutically acceptable salt thereof for use as a medicament.

Another aspect of the present invention is a compound or a pharmaceutically acceptable salt thereof according to the present invention for use in a method of treating or preventing diseases and disorders associated with ZAP-70, Syk or JAK3.

Yet another aspect of the present invention is the use of a compound of the present invention or a pharmaceutically acceptable salt thereof for the manufacture of a medicament for the treatment or prophylaxis of diseases and disorders associated with ZAP-70, Syk or JAK3.

According to the present invention, the expression “ZAP-70” or “ZAP-70 kinase” means “zeta chain-associated protein of 70 kDa”. ZAP-70 associates with the zeta chain of the T cell receptor (TCR) and undergoes tyrosine phosphorylation following TCR stimulation. The ZAP-70 gene is located on human chromosome 2q12 and it is expressed in T cells and natural killer (NK) cells.

According to the present invention, the expression “Syk” or “Syk kinase” means “spleen tyrosine kinase”. Syk is a protein-tyrosine kinase that is widely expressed in hematopoietic cells. It is involved in coupling activated immunoreceptors to downstream signaling events that mediate diversecellular responses, including proliferation, differentiation, and phagocytosis.

According to the present invention, the expression “JAK3” or “JAK3 kinase” means “Janus kinase 3”. The gene encoding JAK3 is located on human chromosome 19p13.1 and it is predominantly in hematopoietic cells. JAK3 is a cytoplasmic protein tyrosine kinase that associates with the gamma-chain of the interleukin 2 (IL-2) receptor. This chain also serves as a component for the receptors of several lymphotropic cytokines, including interleukins IL-4, IL-7, IL-9, IL-15 and IL-21.

Moreover, according to the present invention, the expression “JAK3” or “JAK3 kinase” includes mutant forms of JAK3, preferably JAK3 mutants found in acute megakaryoblastic leukemia (AMKL) patients. More preferred, these mutants are single amino acid mutations. Activating JAK3 mutations were observed in acute megakaryoblastic leukemia (AMKL) patients (Walters et al., 2006. Cancer Cell 10(1):65-75). Therefore, in a preferred embodiment, the expression “JAK” also includes a JAK3 protein having a V7221 or P132T mutation.

Yet another aspect of the present invention is a compound or a pharmaceutically acceptable salt thereof according to the present invention for use in a method of treating or preventing immunological, inflammatory, autoimmune, allergic disorders, or immunologically-mediated diseases.

Yet another aspect of the present invention is the use of a compound of the present invention or a pharmaceutically acceptable salt thereof for the manufacture of a medicament for the treatment or prophylaxis of immunological, inflammatory, autoimmune, allergic disorders, or immunologically-mediated diseases.

More specifically, preferred disorders are acute or chronic inflammation; rheumatoid arthritis; multiple sclerosis; psoriasis; Crohn's disease; ulcerative colitis; systemic lupus erythematosus; asthma; chronic obstructive pulmonary disease (COPD); allergic rhinitis; allograft transplant rejection; graft-versus-host disease; dry eye disorder; or uveitis.

Quite more preferred are rheumatoid arthritis; multiple sclerosis; psoriasis; Crohn's disease; ulcerative colitis; systemic lupus erythematosus; allograft transplant rejection; or graft-versus-host disease.

Rheumatoid arthritis (RA) is a chronic progressive, debilitating inflammatory disease that affects approximately 1% of the world's population. RA is a symmetric polyarticular arthritis that primarily affects the small joints of the hands and feet. In addition to inflammation in the synovium, the joint lining, the aggressive front of tissue called pannus invades and destroys local articular structures (Firestein 2003, Nature 423:356-361).

Multiple sclerosis (MS) is an inflammatory and demyelating neurological disease. It has been considered as an autoimmune disorder mediated by CD4+ type 1 T helper cells, but recent studies indicated a role of other immune cells (Hemmer et al., 2002, Nat. Rev. Neuroscience 3, 291-301).

Psoriasis is a chronic inflammatory dermatosis that affects approximately 2% of the population. It is characterized by red, scaly skin patches that are usually found on the scalp, elbows, and knees, and may be associated with severe arthritis. The lesions are caused by abnormal keratinocyte proliferation and infiltration of inflammatory cells into the dermis and epidermis (Schön et al., 2005, New Engl. J. Med. 352:1899-1912).

Inflammatory bowel disease (IBD) is characterized by a chronic relapsing intestinal inflammation. IBD is subdivided into Crohn's disease and ulcerative colitis phenotypes. Crohn disease involves most frequently the terminal ileum and colon, is transmural and discontinuous. In contrast, in ulcerative colitis, the inflammation is continuous and limited to rectal and colonic mucosal layers. In approximately 10% of cases confined to the rectum and colon, definitive classification of Crohn disease or ulcerative colitis cannot be made and are designated ‘indeterminate colitis.’ Both diseases include extraintestinal inflammation of the skin, eyes, or joints (Asakura et al., 2007, World J. Gastroenterol. 13(15):2145-2149). Systemic lupus erythematosus (SLE) is a chronic inflammatory disease generated by T cell-mediated B-cell activation, which results in glomerulonephritis and renal failure. Human SLE is characterized at early stages by the expansion of long-lasting autoreactive CD4′ memory cells (D'Cruz et al., 2007, Lancet 369(9561):587-596).

Asthma is a complex syndrome with many clinical phenotypes in both adults and children. Its major characteristics include a variable degree of air flow obstruction, bronchial hyperresponsiveness, and airway inflammation (Busse and Lemanske, 2001, N. Engl. J. Med. 344:350-362).

Chronic obstructive pulmonary disease (COPD) is characterized by inflammation, airflow limitation that is not fully reversible, and a gradual loss of lung function. In COPD, chronic inhalation of irritants causes an abnormal inflammatory response, remodeling of the airways, and restriction of airflow in the lungs. The inhaled irritant is usually tobacco smoke, but occupational dust and environmental pollution are variably implicated (Shapiro 2005, N. Engl. J. Med. 352, 2016-2019).

Allergic rhinitis (also known as hay fever) is caused by pollens of specific seasonal plants and airborne chemicals or dust particles in patients who are allergic to these substances. It is characterized by sneezing, runny nose and itching eyes. The immune response to an allergen depends on an initial sensitization process and future exposure triggering the allergic response. This process involves several cell types and mediators of the immune system (Rosenwasser 2007, Allergy Asthma Proc. 28(1):10-15).

Immunologically-mediated diseases include rejection of transplanted organs or tissues (allografts) and graft-versus-host disease.

Allogaft transplant rejection includes, without limitation, acute and chronic allograft rejection following for example transplantation of kidney, heart, liver, lung, bone marrow, skin and cornea. It is known that T cells play a central role in the specific immune response of allograft rejection. Strategies to prevent T cell activation are expected to be useful for immunosuppression (Perico and Remuzzi, 1997. Drugs 54(4):533-570).

Graft-versus-host disease (GVDH) is a major complication in allogeneic bone marrow transplantation. GVDH is caused by donor T cells that recognize and react o recipient differences in the histocompatibility complex system, resulting in significant morbidity and mortality (Riddell and Appelbaum, 2007, PLoS Medicine 4 (7):1174-1177).

Dry eye syndrome (DES, also known as keratoconjunctivitis sicca) is one of the most common problems treated by eye physicians. Sometimes DES is referred to as dysfunctional tear syndrome (Jackson, 2009. Canadian Journal Ophthalmology 44(4), 385-394). DES affects up to 10% of the population between the ages of 20 to 45 years, with this percentage increasing with age. Although a wide variety of artificial tear products are available, these products provide only transitory relief of symptoms. As such, there is a need for agents, compositions and therapeutic methods to treat dry eye.

As used herein, “dry eye disorder” is intended to encompass the disease states summarized in a recent official report of the Dry Eye Workshop (DEWS), which defined dry eye as “a multifactorial disease of the tears and ocular surface that results in symptoms of discomfort, visual disturbance, and tear film instability with potential damage to the ocular surface. It is accompanied by increased osmolality of the tear film and inflammation of the ocular surface.”(Lemp, 2007. “The Definition and Classification of Dry Eye Disease: Report of the Definition and Classification Subcommittee of the International Dry Eye Workshop”, The Ocular Surface, 5(2), 75-92). Dry eye is also sometimes referred to as keratoconjunctivitis sicca. In some embodiments, the treatment of the dry eye disorder involves ameliorating a particular symptom of dry eye disorder, such as eye discomfort, visual disturbance, tear film instability, tear hyperosmolarity, and inflammation of the ocular surface.

As summarized in the DEWS report, dry eye can be classified into two different classes: aqueous tear-deficient dry eye and evaporative dry eye, which in turn encompass various subclasses. Accordingly, in some embodiments, the dry eye disorder is aqueous tear-deficient dry eye (ADDE). In further embodiments, the dry eye disorder is evaporative dry eye. In further embodiments, the dry eye disorder is selected from any of the subclasses of ADDE or evaporative dry eye disorder, or appropriate combinations thereof. As noted by the author of the DEWS report, however, the various classes and subclasses are not mutually exclusive. Hence, dry eye can occur via different mechanism in different subclasses or a dry eye disease state originating in one subclass can lead to events that cause dry eye by a mechanism in another subclass.

The first class of dry eye, aqueous tear-deficient dry eye (ADDE), is also known as tear deficient dry eye and lacrimal tear deficiency. In ADDE, dry eye is believed to be due to a failure of lacrimal tear secretion. While not wishing to be bound by any theory, it is believed that dryness results from reduced lacrimal tear secretion and volume, causing tear hyperosmolarity. Tear film hyperosmolarity can cause hyperosmolarity of the ocular surface epithelial cells, stimulating inflammatory events involving various kinases and signaling pathways.

Two subclasses of ADDE are Sjogren syndrome dry eye (SSDE), where the lacrimal glands are targeted by an autoimmune process, and non-Sjogren syndrome dry eye (NSSDE). Accordingly, in some embodiments, the eye disorder is SSDE. In other embodiments, dry eye disorder is non-Sjogren syndrome dry eye. In SSDE, it is believed that activated T-cells can infiltrate the lacrimal glands, causing cell death of acinar and ductular cells and hyposecretion of tears. The effects of locally released cytokines or circulating antibodies can amplify the effects of hyposecretion. The two major forms of SSDE are primary and secondary forms. Primary SS can occur in combination with dry mouth (xerostomia). Secondary SSDE occurs with the symptoms of primary SSDE together with an autoimmune connective disease such as rheumatoid arthritis (RA), systemic lupus erythematosis, polyarteritis nodosa, Wegener's granulomatosis, systemic sclerosis, primary bilary sclerosis, or mixed connective tissue disease. Diagnostic criteria for each of these connective diseases is known in the art. Further, primary SSDE may be associated with systemic manifestations of disease which may involve the lungs, kidneys, liver, blood vessels and joints.

In NSSDE, the systemic autoimmune characteristics of Sjogren syndrome dry eye are excluded. Forms of NSSDE include primary lacrimal gland deficiencies (including age-related dry eye, congenital alacrima, and familial dysautonomia), secondary lacrimal deficiencies (including inflammatory infiltration of the lacrimal gland by sarcoid granulomata, lymphomatous cells, and AIDS related T-cells; that associated with graft versus host disease; and that resulting from lacrimal gland ablation or lacrimal gland denervation), obstruction of the lacrimal gland ducts (including that caused by cicatrizing conjunctivitis including trachoma, cicatricial pemphigoid and mucous membrane pemphigoid, erythema multiforme, and chemical or thermal burns), and reflex hyposecretion (including reflex sensory block, such as that associated with contact lens wear, diabetes mellitus, and neurotrophic keratitis, and reflex motor block, including that associated with VII cranial nerve damage, multiple neuromatosis, and exposure to systemic drugs such as antihistamines, beta blockers, antispasmodics, diuretics, tricyclic antidepressants, selective serotonin reuptake inhibitors, and other psychotropic drugs). The second major class of dry eye disorder is evaporative dry eye, which is caused by excessive water loss from the exposed ocular surface in the presence of normal lacrimal secretory function. Intrinsic causes of evaporative dry eye include Meibomian gland dysfunction (MGD) (including that caused by a reduced number of glands due to congenital deficiency acquired-MGD; MGD associated with dystichiasis, dystichiasis lymphedema syndrome, and metaplasia; hypersecretory MGD associated with Meibomian seborrhea, hypersecretory MGD associated with retinoid therapy, primary and secondary obstructive MGD, focal or diffuse obstructive MGD, simple or cicatricial obstructive MGD, atrophic or inflammatory obstructive MGD; Simple MGD primary or secondary to anterior blepharitis, acne rosacea, seborrhoeic dermatitis, ectrodactyly syndrome, Turner syndrome, systemic toxicity from 13-cis retinoic acid, polychlorinated biphenyls, and epinephrine; and cicatricial MGD primary or secondary to chemical burns, pemphigoid, acne rosacea, erythema multiforms, VKC and AKC), disorders of the lid aperture and lid/globe congruity or dynamic (such as that occurring with craniostenosis, endocrine and other forms of proptosis, myopia, and after plastic surgery on the lids), and low blink rate (including that caused by an extrapyramidal disorder such as Parkinson's disease). Extrinsic causes of evaporative dry eye include ocular surface disorders (including xerophthalmia caused by vitamin A deficiency; and that associated with topical drugs and preservatives such as topical anesthesia and benzalkonium chloride), contact lens wear, ocular surface disease (including allergic eye disease), allergic conjunctivitis (including aseasonal allergic conjunctivitis, vernal keratoconjunctivitis, and atopic keratoconjunctivitis), and the use of antihistamines.

Patients in need of treatment of a dry eye disorder can be identified by a variety of diagnostic methods known in the art, including the diagnostic methods summarized in Bron, et al., “Methodologies to Diagnose and Monitor Dry Eye Disease: Report of the Diagnostic Methodology Subcommittee of the International Dry Eye Workshop (2007)”, The Ocular Surface, 5(2), 108-152 (April 2007), which is hereby incorporated herein by reference in its entirety.

In a further aspect, the present invention provides a method of treating conjunctivitis, uveitis (including chronic uveitis), chorioditis, retinitis, cyclitis, sclieritis, episcleritis, or iritis; treating inflammation or pain related to corneal transplant, LASIK (laser assisted in situ keratomileusis), photorefractive keratectomy, or LASEK (laser assisted sub-epithelial keratomileusis); inhibiting loss of visual acuity related to corneal transplant, LASIK, photorefractive keratectomy, or LASEK; or inhibiting transplant rejection in a patient in need thereof, comprising administering to the patient a therapeutically effective amount of an agent, or pharmaceutically acceptable salt thereof. In some embodiments, the agent is administered preoperatively to a patient about to undergo a procedure selected from corneal transplant, LASIK, photorefractive keratectomy, and LASEK. In some embodiments, the agent suppresses or lessens inflammation or pain during and after the procedure. In some embodiments, the agent is administered about 1 day to about 2 days prior to the procedure. In some embodiments, the agent is administered postoperatively to a patient who has undergone a procedure selected from corneal transplant, LASIK, photorefractive keratectomy, and LASEK. In some embodiments, inhibiting loss of visual acuity means lessening the loss of visual acuity. In some embodiments, the postoperative or preoperative treatment lessens the amount of scarring and fibrous deposits following the procedure. In some embodiments, inhibiting loss of visual acuity means that the patient retains visual acuity. In some embodiments, inhibiting transplant rejection means that the agent is immunosuppressive, thereby preventing total rejection of the corneal transplant.

Uveitis is the most common form of intraocular inflammation and remains a significant cause of visual loss. Current treatments for uveitis employs systemic medications that have severe side effects and are globally immunosuppressive. Clinically, chronic progressive or relapsing forms of non-infectious uveitis are treated with topical and/or systemic corticosteroids. In addition, macrolides such as cyclosporine and rapamycin are used, and in some cases cytotoxic agents such as cyclophosphamide and chlorambucil, and antimetabolites such as azathioprine, methotrexate, and leflunomide (Srivastava et al., 2010. Uveitis: Mechanisms and recent advances in therapy. Clinica Chimica Acta, doi:10.1016/j.cca.2010.04.017).

Further eye diseases, combination treatments and route of administration are described for example in WO-A 2010/039939, which is hereby incorporated herein by reference.

Another aspect of the present invention is a method for treating, controlling, delaying or preventing in a mammalian patient in need of the treatment of one or more conditions selected from the group consisting of diseases and disorders associated with ZAP-70, Syk or JAK3 wherein the method comprises the administration to said patient a therapeutically effective amount of a compound according to present invention or a pharmaceutically acceptable salt thereof.

Yet another aspect of the present invention is a method for treating, controlling, delaying or preventing in a mammalian patient in need of the treatment of one or more conditions selected from the group consisting of immunological, inflammatory, autoimmune, allergic disorders, and immunologically-mediated diseases, wherein the method comprises the administration to said patient a therapeutically effective amount of a compound according to the present invention or a pharmaceutically acceptable salt thereof.

More specifically the one or more conditions are selected from the group consisting of immunological, inflammatory, autoimmune, allergic disorders, or immunologically-mediated diseases, especially acute or chronic inflammation; rheumatoid arthritis; multiple sclerosis; psoriasis; Crohn's disease; ulcerative colitis; systemic lupus erythematosus; asthma; chronic obstructive pulmonary disease (COPD); allergic rhinitis; allograft transplant rejection; graft-versus-host disease; dry eye disorder; or uveitis.

As used herein, the term “treating” or “treatment” is intended to refer to all processes, wherein there may be a slowing, interrupting, arresting, or stopping of the progression of a disease, but does not necessarily indicate a total elimination of all symptoms.

The compounds of the present invention may be further characterized by determining whether they have an effect on ZAP-70, Syk or JAK3 activity, for example on its kinase activity (Isakov et al., 1996, J. Biol. Chem. 271(26), 15753-15761; Moffat et al., 1999, Bioorg. Med. Chem. Letters 9, 3351-3356; Changelian et al., 2003, Science 302(5646):875-888 and online supplement; Yang et al., 2007. Bioorg. Med. Chem. Letters 17(2): 326-331).

The compounds of the present invention may also be characterized by measuring whether they have an effect on T cell receptor (TCR) signaling in a cell based assay using a T cell line or primary T cells. Cellular activation that is initiated by TCR signaling occurs as a result of a series of molecular events that include tyrosine phosphorylation of the CD3 zeta (CD3ζ) chain, recruitment of ZAP-70, phosphorylation of phospholipase gamma 1 (PLCγ1), inositol 1,4,5-triphosphate production, release of calcium stores from the endoplasmic reticulum to the cytoplasm, secretion of cytokines (for example Interleukin 2, IL-2), and cell proliferation.

The effect of compounds on tyrosine phosphorylation of PLCγ1 in Jurkat T cells following stimulation with anti-CD3 antibody can be examined by immunoprecipitation of PLCγ1 with an anti-PLCγ1 antibody and probing with an anti-phosphotyrosine specific antibody (e.g. antibody 4G10; Lin et al., 2004, Biochemistry 43, 11056-11062). Methods for measuring intracellular calcium release using fluorescent indicators for cytosolic calcium after TCR stimulation have been described (Meinl et al., 2000, J. Immunol. 165(7):3578-3583).

To evaluate the effect of compounds on the secretion of IL-2 T cells are stimulated with an anti-CD-3 antibody and incubated with various compound concentrations, then the concentration of IL-2 is measured in the cell-free media by an enzyme-linked immunosorbent assay (ELISA). A similar approach can be used to determine whether the compounds show activity in vivo. Mice are dosed with the compound of interest (e.g. by orally administration) followed by stimulation by intravenous injection of an anti-CD3 antibody. Serum is collected and the level of cytokines (e.g. IL-2) is measured in an ELISA (Lin et al., 2004, Biochemistry 43, 11056-11062).

A cell-based assay (TF-1 cell proliferation) was described to assess the inhibitory activity of small molecule drugs toward JAK2 or JAK3-dependent signal transduction (Chen et al., 2006. Bioorg. Med. Chem. Letters 16(21): 5633-5638).

The present invention provides pharmaceutical compositions comprising a compound of formula (I) or a pharmaceutically acceptable salt thereof as active ingredient together with a pharmaceutically acceptable carrier, optionally in combination with one or more other pharmaceutical compositions.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

A pharmaceutical composition of the present invention may comprise one or more additional compounds as active ingredients like one or more compounds of formula (I) not being the first compound in the composition or ZAP-70, Syk or JAK3 inhibitors.

Other active ingredients for use in combination with other therapies for the treatment of immune, inflammatory, allergic disorders may include steroids, leukotriene antagonists, cyclosporine or rapamycin.

Other active ingredients include: immunosuppresants such as amtolmetin guacil, mizoribine and rimexolone; anti-TNFα agents such as etanercept, infliximab, Adalimumab, Anakinra, Abatacept, Rituximab; tyrosine kinase inhibitors such as leflunomide; kallikrein antagonists such as subreum; interleukin 11 agonists such as oprelvekin; interferon beta 1 agonists; hyaluronic acid agonists such as NRD-101 (Aventis); interleukin 1 receptor antagonists such as anakinra; CD8 antagonists such as amiprilose hydrochloride; beta amyloid precursor protein antagonists such as reumacon; matrix metalloprotease inhibitors such as cipemastat and other disease modifying anti-rheumatic drugs (DMARDs) such as methotrexate, sulphasalazine, cyclosporin A, hydroxychoroquine, auranofin, aurothioglucose, gold sodium thiomalate and penicillamine.

The individual compounds of such combinations may be administered either sequentially in separate pharmaceutical compositions as well as simultaneously in combined pharmaceutical compositions.

The pharmaceutical compositions of the present invention include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally, for example, as liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula (I) may also be administered parenterally. Solutions or suspensions of these active compounds can be prepared in water suitably mixed with a surfactant such as hydroxypropyl-cellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula (I) are administered orally.

The effective dosage of active ingredient employed may vary depending on the particular compound employed, the mode of administration, the condition being treated and the severity of the condition being treated. Such dosage may be ascertained readily by a person skilled in the art.

A general route for the preparation of compounds according to present invention is outlined in Schemes 1 and 2.

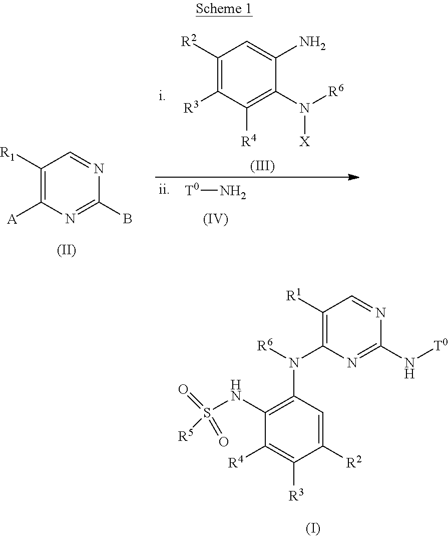

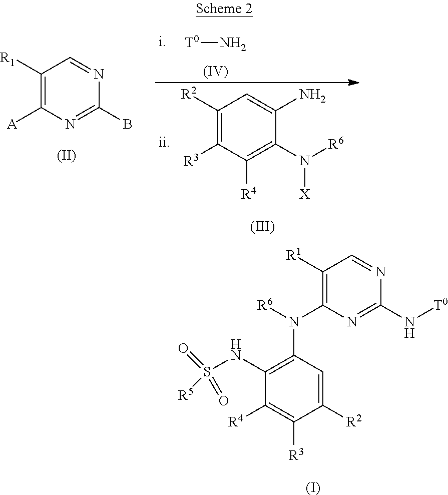

Compounds of formula (I) can be formed from compounds (II), (III) and (IV) by reacting (II) with (III) then reacting the resultant adduct with (IV) according to Scheme 1. Alternatively (I) may be formed by the reaction of (II) with (IV) then reacting the resultant adduct with (III) according to Scheme 2. The person skilled in the art would understand that the order of events would depend on the conditions of the reaction and the nature of (I), (II), (III) and (IV). Compounds (II), (III) and (IV) are either commercially available or can be made by those skilled in the art. A wide range of solvents are optionally employed for these reactions, including protic solvents such as alcohols, or polar aprotic solvents such as dimethylsulfoxide, DMF, acetonitrile, dioxane, THF. The reactions can optionally be promoted by the addition of a base which include but are not limited to amine bases such as triethylamine and DIPEA; or metal carbonates. The reactions can be optionally promoted by acids including mineral acids such as hydrogen chloride; organic acids and Lewis acids such as zinc (II) chloride. The reactions can be optionally promoted by a transition metal catalyst such as a palladium or copper catalyst, in conjunction with a suitable ligand such as a phosphine ligand. These reactions are typically performed between −78° C. and 160° C. depending on the nature of (I), (II), (III) and (IV). A and B are suitable leaving groups such as halogens, O—C₁₋₆ alkyl, N—C₁₋₆ alkyl, N(C₁₋₆ alkyl)₂, S—C₁₋₆ alkyl and SO₂—C₁₋₆ alkyl.

In Schemes 1 and 2, X is S(O)₂R⁵ or H or a suitable protecting group known to those skilled in the art. If X in (III) is other than S(O)₂R⁵ then intermediate steps will be inserted in Scheme 1 or Scheme 2 to facilitate the conversion of X to give compounds of formula (I). The person skilled in the art will recognise that these insertions might occur after either step (i) or step (ii). If X is a protecting group then the removal of the protecting group precedes the conversion of X to S(O)₂R⁵. This conversion can be introduced by reacting the intermediate wherein X is H, with a compound GS(O)₂R⁵ wherein G is a suitable leaving group. Commonly G is chlorine or oxazolidinone. The skilled person would recognise that a wide range of solvents may be employed to effect this process and that the addition of a base may be beneficial. In one embodiment, DCM is used as a solvent and triethylamine is used as a base. In another embodiment, pyridine is used as base and solvent. Compounds of formula GS(O)₂R⁵ are either commercially available or can be prepared by those skilled in the art.

In one embodiment, a compound of formula (II) is reacted with a compound of formula (III) in the presence of an amine base, such as DIPEA; in a protic solvent, such as IPA; at a temperature above −20° C., such as 80° C. The adduct is isolated by means known to those skilled in the art, then reacted with a compound of formula GS(O)₂R⁵, such as a sulfonyl chloride; in a basic solvent, such as pyridine; at a temperature above −20° C., such as 20° C. The adduct is isolated by means known to those skilled in the art, then reacted with a compound of formula (IV) in the presence of a mineral acid, such as hydrogen chloride; in a protic solvent, such as IPA; at a temperature above 20° C., such as 80° C. to yield a compound of formula (I). In this embodiment it is conceivable that (I) is isolated in a salt form, such as a hydrochloride salt. In another embodiment, a compound of formula (I) is formed from a compound of formula (IV) using a transition metal catalyst, such as palladium acetate; in the presence of a ligand, such as Xantphos; in a polar aprotic solvent, such as dioxane; at a temperature above 20° C., such as 160° C. to yield a compound of formula (I).

Accordingly, another aspect of the present invention is a method for the preparation of a compound of formula (I) of the present invention, comprising the steps of
(a) reacting a compound of formula (II)

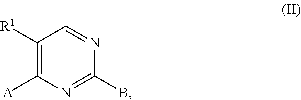

wherein R¹ has the meaning as indicated above and A, B are suitable leaving groups with one of the compounds of formula (III) or (IV)

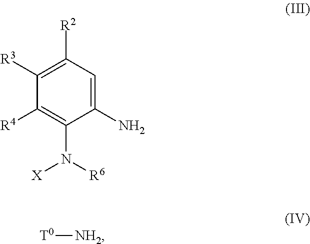

wherein X is S(O)₂R⁵; or H and wherein R², R³, R⁴, R⁵, R⁶, T⁰ have the meaning as indicated above and;
(b) reacting the resulting product from step (a) with the other compound of formula (III) or (IV) to yield a compound of formula (I) when X is S(O)₂R⁵; or
(c) reacting the resulting product of step (b) when X is H with a compound of formula R⁵S(O)₂C1 to yield a compound of formula (I).

It will be appreciated that novel intermediates described herein form another embodiment of the present invention.

### EXAMPLES

### Analytical Methods

NMR spectra were obtained on a Bruker dpx400. LCMS was carried out on an Agilent 1100 using a ZORBAX® SB-C18, 4.6×150 mm, 5 microns or ZORBAX® SB-C18, 4.6×75 mm, 3.5 micron column. Column flow was 1 mL/min and solvents used were water and acetonitrile (0.1% formic acid) with an injection volume of 10 uL. Wavelengths were 254 and 210 nm. Methods are described below.

### Method A

Column: Gemini C18, 3×30 mm, 3 microns Flow: 1.2 mL/min. Gradient: Table 1

TABLE 1

| Time (min) | Water | Acetonitrile |
|---|---|---|
| 0 | 95 | 5 |
| 3 | 5 | 95 |
| 4.5 | 5 | 95 |
| 4.6 | 95 | 5 |

| | |
|---|---|
| 5.00 | STOP |

### Method B

Column: ZORBAX® SB-C18, 4.6×150 mm, 5 microns. Flow: 1 mL/min. Gradient: Table 2

TABLE 2

| Time (min) | Water | Acetonitrile |
|---|---|---|
| 0 | 95 | 5 |
| 11 | 5 | 95 |
| 13 | 5 | 95 |
| 13.01 | 95 | 5 |

| | |
|---|---|
| 14.00 | STOP |

### Method C

As Method A but with 0.1% ammonium hydroxide instead of 0.1% formic acid.

### Abbreviations

TABLE 3

| | |
|---|---|
| IPA | iso-propyl alcohol |
| THF | tetrahydrofuran |
| DIPEA | di-iso-propylethylamine |
| Xantphos | 9.9-Dimethyl-4,5-bis-(diophenylphosphino)xanthene |
| Me | methyl |
| Et | ethyl |
| ⁱPr | iso-propyl |
| Ph | phenyl |
| h | hour |
| min | minute |
| cat. | catalytic |
| M | molar |
| NMR | nuclear magnetic resonance |
| DMSO | dimethylsulfoxide |
| MeOD | deuterated methanol (d₄-methanol) |
| s | singlet |
| d | doublet |
| dd | doublet doublet |
| td | triplet doublet |
| br | broad |
| q | quartet |
| t | triplet |
| m | multiplet |
| ES+ | electrospray positive ionisation |
| RT | retention time |

### Example 1

### N-(2-(5-fluoro-2-(1-methyl-1H-pyrazol-3-ylamino)pyrimidin-4-ylamino)phenyl)methanesulfonamide

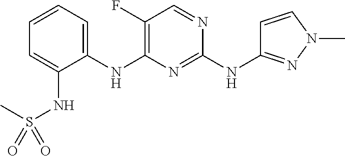

### Step (i)

### N1-(2-chloro-5-fluoropyrimidin-4-yl)benzene-1,2-diamine

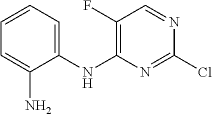

A mixture of 2,4-dichloro-5-fluoropyrimidine (10.0 g, 0.06 mol), o-phenylenediamine (7.1 g, 0.066 mol) and DIPEA (20.8 mL, 0.12 mol) in n-butanol (80 mL) was stirred at 110° C. for 16 h then concentrated in vacuo and slurried with 0.1 M hydrochloric acid (20 mL). The solid was collected at the pump, washed with water (2×20 mL), n-butanol (30 mL) and diethyl ether (2×30 mL), then dried under vacuum to afford N1-(2-chloro-5-fluoropyrimidin-4-yl)benzene-1,2-diamine as a colourless powder (10.8 g, 71%). ¹H NMR (d₆-DMSO) δ 9.31 (br s, 1H), 8.18 (d, 1H), 6.99-7.03 (m, 2H), 6.74-6.76 (m, 1H), 6.54-6.58 (m, 1H), 5.04 (br s, 2H); LCMS method A, (ES+) 239, 241, RT=1.90 min.

### Step (ii)

### N-(2-(2-chloro-5-fluoropyrimidin-4-ylamino)phenyl)methanesulfonamide

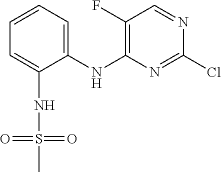

Methanesulfonyl chloride (0.54 mL, 6.93 mmol) was added dropwise to a solution of N1-(2-chloro-5-fluoropyrimidin-4-yl)benzene-1,2-diamine (1.5 g, 6.30 mmol) in pyridine (15 mL) at 0° C. then warmed to room temperature. After 18 h the mixture was diluted with water (25 mL) and extracted with ethyl acetate (25 mL). The separated organic layer was washed with 2M hydrochloric acid (2×25 mL) and brine (25 mL), dried (MgSO₄) and concentrated in vacuo to afford N-(2-(2-chloro-5-fluoropyrimidin-4-ylamino)phenyl)methanesulfonamide as a beige solid (1.45 g, 72%). ¹H NMR (d₆-DMSO) δ 9.41 (br s, 1H), 9.25 (s, 1H), 8.30 (d, 1H), 7.47-7.52 (m, 2H), 7.32 (t, 1H), 7.25 (t, 1H), 2.99 (s, 3H); LCMS method A, (ES+) 316, RT=2.26 min.

### Step (iii)

### N-(2-(5-fluoro-2-(1-methyl-1H-pyrazol-3-ylamino)pyrimidin-4-ylamino)phenyl)methanesulfonamide

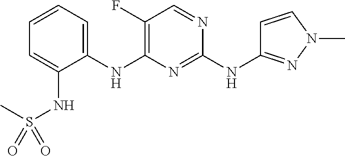

A mixture of N-(2-(2-chloro-5-fluoropyrimidin-4-ylamino)phenyl)methanesulfonamide (100 mg, 0.32 mmol), 3-amino-1-methyl-M-pyrazole (30 mg, 0.32 mmol) and HCl (4 M in dioxane, cat.) in IPA (5 mL) was stirred at 120° C. for 4 h in a microwave reactor. On standing the mixture yielded a precipitate which was collected at the pump, washed with cold IPA then purified by preparative HPLC to afford the title product as a beige solid. ¹H NMR: (d₆-DMSO) δ 9.15 (s, 1H), 8.48 (s, 1H), 8.01 (s, 1H), 7.94 (d, 1H), 7.83 (d, 1H), 7.27 (m, 2H), 7.08 (m, 2H), 6.04 (s, 1H), 3.55 (s, 3H), 2.76 (s, 3H); LCMS method A, (ES+) 378, RT=1.74 min.

### Example 2

### N-(2-(5-chloro-2-(3-methyl-1H-pyrazol-4-ylamino)pyrimidin-4-ylamino)-5-methoxyphenyl)methanesulfonamide

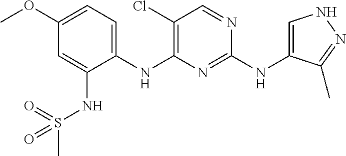

A mixture of N-(2-(2,5-dichloropyrimidin-4-ylamino)-5-methoxyphenyl)methane sulfonamide (150 mg, 0.41 mmol), prepared by the method described in Example 1 steps (i) and (ii), 5-methyl-M-pyrazol-4-amine dihydrochloride semi-hydrate (73 mg, 0.41 mmol), palladium acetate (2 mg, 0.008 mmol), Xantphos (7 mg, 0.012 mmol) and cesium carbonate (269 mg, 0.83 mmol) in 1,4-dioxane (2 mL) was stirred at 160° C. for 5 h in a microwave reactor then diluted with water (5 mL) and extracted with ethyl acetate (5 mL). The organic layer was washed with brine (5 mL), dried (MgSO₄), concentrated in vacuo and purified by preparative HPLC to afford the title compound (35 mg, 20%). ¹H NMR (MeOD) δ 8.15 (s, 1H), 7.92 (s, 1H), 7.57 (d, 1H), 7.42 (br s, 1H), 7.02 (d, 1H), 6.87 (dd, 1H), 3.84 (s, 3H), 2.90 (br s, 3H), 2.10 (s, 3H); LCMS method B, (ES+) 424, RT=5.60 min.

Examples 3-42 can be synthesised by the procedures outlined in Examples 1 and 2, using the appropriate reactants.

### Example 3

### N-(2-(5-chloro-2-(isoxazol-3-ylamino)pyrimidin-4-ylamino)-5-methoxyphenyl)methanesulfonamide

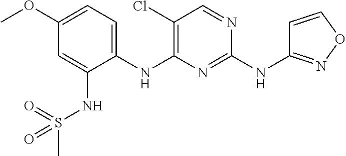

¹H NMR (d₆-DMSO) δ 10.14 (s, 1H), 9.21 (br s, 1H), 8.56 (d, 1H), 8.52 (br s, 1H), 8.14 (s, 1H), 7.62 (d, 1H), 7.00 (d, 1H), 6.86 (d, 1H), 6.50 (s, 1H), 3.79 (s, 3H), 2.91 (s, 3H); LCMS method A, (ES+) 411, RT=2.30 min.

### Example 4

### N-(2-(5-chloro-2-(1-methyl-1H-pyrazol-3-ylamino)pyrimidin-4-ylamino)-5-methoxyphenyl)methanesulfonamide

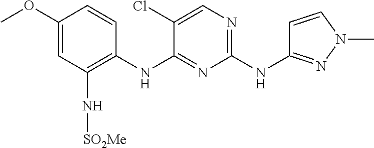

¹H NMR (d₆-DMSO) δ 9.34 (s, 1H), 9.19 (br s, 1H), 8.34 (s, 1H), 8.05 (s, 1H), 7.66 (d, 1H), 7.36 (d, 1H), 6.98 (d, 1H), 6.86 (dd, 1H), 6.02 (br s, 1H), 3.79 (s, 3H), 3.67 (s, 3H), 2.89 (s, 3H); LCMS method B, (ES+) 424, RT=6.30 min.

### Example 5

### N-(2-(5-chloro-2-(1-methyl-1H-pyrazol-5-ylamino)pyrimidin-4-ylamino)-5-methoxyphenyl)methanesulfonamide

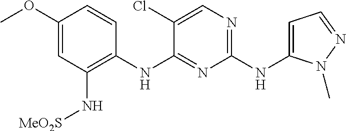

¹H NMR (MeOD) δ 7.98 (s, 1H), 7.55 (d, 1H), 7.34 (d, 1H), 6.99 (d, 1H), 6.82 (dd, 1H), 6.06 (d, 1H), 3.82 (s, 3H), 3.58 (s, 3H), 2.91 (s, 3H); LCMS method B, (ES+) 424, RT=7.65 min.

### Example 6

### N-(2-(5-chloro-2-(1-methyl-1H-pyrazol-4-ylamino)pyrimidin-4-ylamino)-5-methoxyphenyl)methanesulfonamide

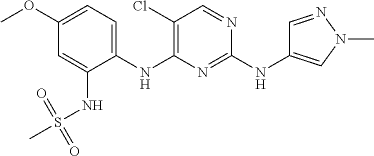

¹H NMR (d₆-DMSO) δ 9.16 (br s, 1H), 8.34 (s, 1H), 8.03 (s, 1H), 7.45 (br s, 1H), 7.17-6.90 (m, 4H), 3.80 (s, 3H), 3.59 (s, 3H), 2.87 (s, 2H); LCMS method B, (ES+) 424, RT=6.34 min.

### Example 7

### N-(2-(5-fluoro-2-(1-methyl-1H-pyrazol-3-ylamino)pyrimidin-4-ylamino)-5-methoxyphenyl)methanesulfonamide

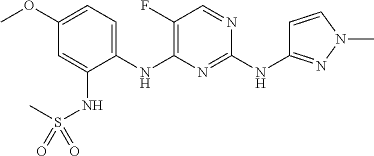

¹H NMR: (d₆-DMSO) δ 10.94 (br s, 1H), 10.30 (s, 1H), 9.32 (s, 1H), 8.27 (d, 1H), 7.63 (s, 1H), 7.33 (d, 1H), 7.12 (d, 1H), 6.85 (dd, 1H), 5.89 (s, 1H), 3.80 (s, 6H), 2.99 (s, 3H); LCMS method A, (ES+) 408, RT=1.83 min.

### Example 8

### N-(2-(5-chloro-2-(1,3-dimethyl-1H-pyrazol-5-ylamino)pyrimidin-4-ylamino)-5-methoxyphenyl)methanesulfonamide

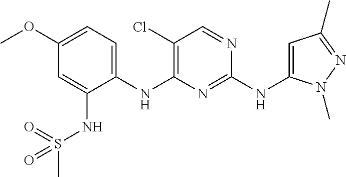

¹H NMR (MeOD) δ 8.16 (s, 1H), 7.99 (s, 1H), 7.52 (d, 1H), 7.03 (d, 1H), 6.85 (dd, 1H), 5.81 (s, 1H), 3.83 (s, 3H), 3.52 (s, 3H), 2.89 (s, 3H), 2.13 (s, 3H); LCMS method B, (ES+) 438, RT=7.52 min.

### Example 9

### N-(2-(5-chloro-2-(3-cyclopropyl-1-methyl-1H-pyrazol-5-ylamino)pyrimidin-4-ylamino)-5-methoxyphenyl)methanesulfonamide

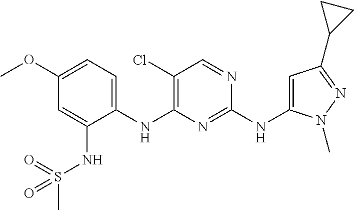

¹H NMR (MeOD) δ 8.11 (br s, 2H), 8.09 (br q, 1H), 8.00 (s, 1H), 7.50 (d, 1H), 7.04 (d, 1H), 6.85 (dd, 1H), 5.66 (s, 1H), 3.84 (s, 3H), 3.52 (s, 3H), 2.88 (s, 3H), 1.80-1.74 (m, 1H), 0.88-0.83 (m, 2H), 0.58-0.54 (m, 2H); LCMS method B, (ES+) 465, RT=8.18 min.

### Example 10

### N-(2-(2-(3-tert-butyl-1-methyl-1H-pyrazol-5-ylamino)-5-chloropyrimidin-4-ylamino)-5-methoxyphenyl)methanesulfonamide

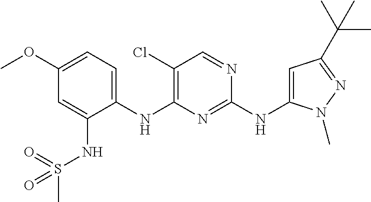

¹H NMR (MeOD) δ 8.00 (s, 1H), 7.50 (d, 1H), 7.02 (d, 1H), 6.83 (dd, 1H), 5.89 (s, 1H), 3.82 (s, 3H), 3.54 (s, 3H), 2.87 (s, 3H), 1.23 (s, 9H); LCMS method B, (ES+) 480, RT=8.98 min.

### Example 11

### N-(2-(5-chloro-2-(3-isopropyl-1-methyl-1H-pyrazol-5-ylamino)pyrimidin-4-ylamino)-5-methoxyphenyl)methanesulfonamide

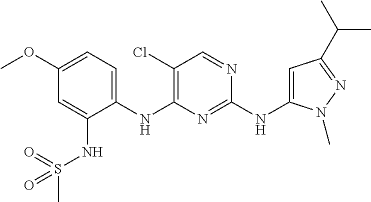

¹H NMR (MeOD) δ 8.09 (s, 2H), 8.00 (s, 1H), 7.50 (d, 1H), 7.03 (d, 1H), 6.83 (dd, 1H), 5.84 (s, 1H), 3.82 (s, 3H), 3.72 (br d, 1H), 3.54 (s, 3H), 2.88 (s, 3H), 2.80 (septet, 1H), 1.18 (d, 6H); LCMS method B, (ES+) 467, RT=8.45 min.

### Example 12

### N-(2-(5-chloro-2-(1-ethyl-3-methyl-1H-pyrazol-5-ylamino)pyrimidin-4-ylamino)-5-methoxyphenyl)methanesulfonamide

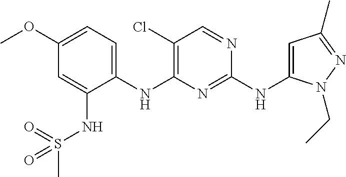

¹H NMR (MeOD) δ 8.11 (s, 1H), 7.98 (s, 1H), 7.53 (d, 1H), 7.02 (d, 1H), 6.84 (dd, 1H), 5.82 (s, 1H), 3.90 (q, 3H), 3.83 (s, 3H), 2.89 (s, 3H), 2.15 (s, 3H), 1.23 (t, 3H); LCMS method B, (ES+) 453, RT=7.88 min.

### Example 13

### N-(2-(5-chloro-2-(1,3,5-trimethyl-1H-pyrazol-4-ylamino)pyrimidin-4-ylamino)-5-methoxyphenyl)methanesulfonamide

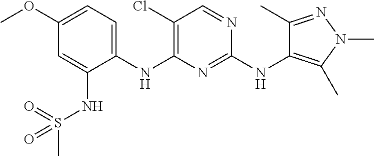

¹H NMR (MeOD) δ 8.14 (s, 1H), 7.87 (br s, 1H), 7.54 (br s, 1H), 6.97 (br s, 1H), 6.82 (br s, 1H), 3.82 (s, 3H), 3.68 (s, 3H), 2.89 (br s, 3H), 1.99 (br s, 3H), 1.96 (br s, 3H); LCMS method B, (ES+) 452, RT=5.87 min.

### Example 14

### N-(2-(5-chloro-2-(1,4-dimethyl-1H-pyrazol-5-ylamino)pyrimidin-4-ylamino)-5-methoxyphenyl)methanesulfonamide

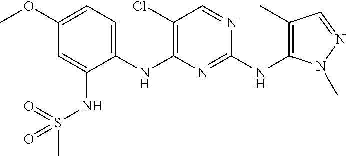

¹H NMR (MeOD) δ 8.16 (s, 1H), 7.95 (s, 1H), 7.55 (d, 1H), 7.27 (s, 1H), 6.95 (d, 1H), 6.79 (dd, 1H), 3.81 (s, 3H), 3.54 (s, 3H), 2.92 (s, 3H), 1.86 (s, 3H); LCMS method B, (ES+) 438, RT=7.78 min.

### Example 15

### N-(2-(5-chloro-2-(3-ethyl-1,4-dimethyl-1H-pyrazol-5-ylamino)pyrimidin-4-ylamino)-5-methoxyphenyl)methanesulfonamide

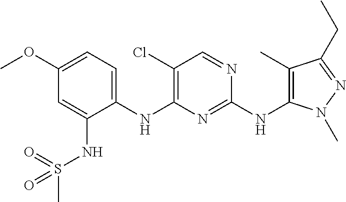

¹H NMR (MeOD) δ 8.22 (s, 1H), 7.95 (s, 1H), 7.52 (d, 1H), 6.95 (br s, 1H), 6.79 (d, 1H), 3.81 (s, 3H), 3.48 (s, 3H), 2.89 (s, 3H), 2.54 (q, 2H), 1.78 (s, 3H), 1.20 (t, 3H); LCMS method B, (ES+) 466, RT=8.27 min.

### Example 16

### N-(2-(5-chloro-2-(1-methyl-1H-pyrazol-3-ylamino)pyrimidin-4-ylamino)phenyl)methanesulfonamide

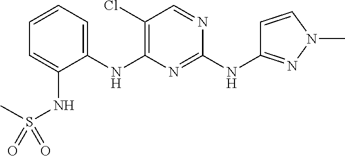

¹H NMR: (d₆-DMSO) δ 9.52 (s, 1H), 9.27 (s, 1H), 8.50 (s, 1H), 8.13 (s, 1H), 8.09 (br s, 1H), 7.39 (m, 2H), 7.32 (t, 1H), 7.23 (t, 1H), 6.14 (s, 1H), 3.71 (s, 3H), 2.95 (s, 3H); LCMS method A, (ES+) 394, RT=1.88 min.

### Example 17

### N-(2-(5-chloro-2-(4-methoxy-1,2,5-oxadiazol-3-ylamino)pyrimidin-4-ylamino)-5-methoxyphenyl)methanesulfonamide

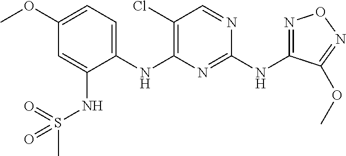

¹H NMR (MeOD) δ 8.09 (s, 1H), 7.80 (d, 1H), 7.00 (d, 1H), 6.89 (dd, 1H), 4.01 (s, 3H), 3.84 (s, 3H), 2.97 (s, 3H); LCMS method B, (ES+) 442, RT=8.95 min.

### Example 18

### N-(2-(5-chloro-2-(1,3-dimethyl-1H-pyrazol-4-ylamino)pyrimidin-4-ylamino)-5-methoxyphenyl)methanesulfonamide

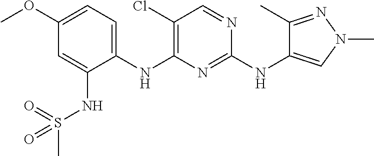

¹H NMR (d₆-DMSO) δ 9.34 (br s, 1H), 8.54 (br s, 1H), 8.38 (s, 1H), 8.01 (s, 1H), 7.53 (br s, 1H), 7.15 (br s, 1H), 7.01 (s, 1H), 6.83 (dd, 1H), 3.78 (s, 3H), 3.55 (br s, 3H), 2.86 (s, 3H), 2.05 (s, 3H); LCMS method B, (ES+) 438, RT=6.22 min.

### Example 19

### N-(2-(5-chloro-2-(3,5-dimethylisoxazol-4-ylamino)pyrimidin-4-ylamino)-5-methoxyphenyl)methanesulfonamide

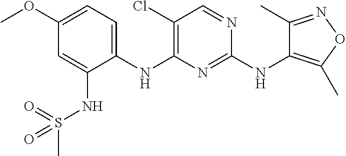

¹H NMR (MeOD) δ 8.09 (s, 2H), 7.93 (s, 1H), 7.55 (d, 1H), 6.97 (d, 1H), 6.82 (d, 1H), 3.82 (s, 3H), 2.92 (s, 3H), 2.18 (s, 3H), 2.01 (s, 3H); LCMS method B, (ES+) 439, RT=7.60 min.

### Example 20

### N-(2-(5-chloro-2-(1-methyl-1H-pyrazol-4-ylamino)pyrimidin-4-ylamino)phenyl)methanesulfonamide

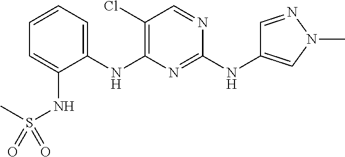

¹H NMR: (d₆-DMSO) δ 10.59 (s, 1H), 9.99 (s, 1H), 9.34 (s, 1H), 8.33 (s, 1H), 7.49 (m, 4H), 7.18 (s, 1H), 6.99 (s, 1H), 3.57 (s, 3H), 2.92 (s, 3H); LCMS method A, (ES+) 394, RT=1.84 min.

### Example 21

### N-(2-(5-fluoro-2-(1-methyl-1H-pyrazol-4-ylamino)pyrimidin-4-ylamino)-5-methoxyphenyl)methanesulfonamide

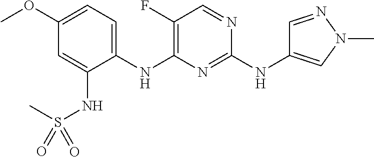

¹H NMR: (d₆-DMSO) δ 9.29 (br s, 1H), 8.98 (s, 1H), 8.56 (s, 1H), 7.99 (d, 1H), 7.43 (d, 1H), 7.18 (s, 1H), 7.08 (s, 1H), 6.89 (d, 1H), 3.80 (s, 3H), 3.61 (s, 3H), 2.88 (s, 3H); LCMS method A, (ES+) 408, RT=1.65 min.

### Example 22

### N-(2-(5-fluoro-2-(1-methyl-1H-pyrazol-4-ylamino)pyrimidin-4-ylamino)phenyl)methanesulfonamide

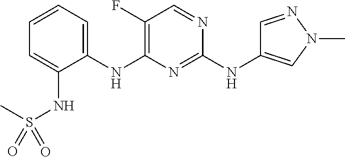

¹H NMR: (d₆-DMSO) δ 9.05 (s, 1H), 8.67 (s, 1H), 8.15 (s, 1H), 8.06 (d, 1H), 7.70 (br s, 1H), 7.48 (d, 1H), 7.24 (m, 3H), 3.65 (s, 3H), 2.90 (s, 3H); LCMS method A, (ES+) 378, RT=1.61 min.

### Example 23

### N-(2-(2-(1H-pyrazol-4-ylamino)-5-fluoropyrimidin-4-ylamino)phenyl)methanesulfonamide

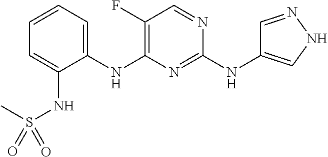

¹H NMR: (d₆-DMSO) δ 10.21 (br s, 2H), 9.35 (s, 1H), 8.24 (br s, 1H), 7.8-7.1 (m, 6H), 2.92 (s, 3H); LCMS method A, (ES+) 364, RT=1.42 min.

### Example 24

### N-(2-(2-(1H-pyrazol-4-ylamino)-5-fluoropyrimidin-4-ylamino)-5-methoxyphenyl)methanesulfonamide

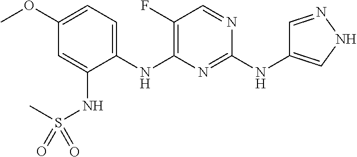

¹H NMR: (d₆-DMSO) δ 10.40 (br s, 1H), 10.24 (br s, 1H), 9.34 (s, 1H), 8.23 (br s, 1H), 7.35 (d, 1H), 7.31 (br s, 1H), 7.11 (d, 1H), 6.86 (dd, 1H), 3.80 (s, 3H); LCMS method A, (ES+) 394, RT=1.52 min.

### Example 25

### N-(2-(2-(1H-pyrazol-4-ylamino)-5-chloropyrimidin-4-ylamino)phenyl)methanesulfonamide

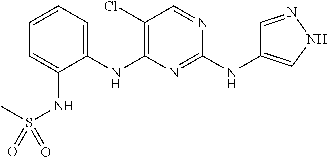

¹H NMR: (d₆-DMSO) δ 10.42 (s, 1H), 9.81 (s, 1H), 9.32 (s, 1H), 8.32 (s, 1H), 7.94-8.00 (m, 6H), 2.91 (s, 3H); LCMS method A, (ES+) 380, RT=1.69 min.

### Example 26

### N-(2-(5-chloro-2-(1-ethyl-4-methyl-1H-pyrazol-5-ylamino)pyrimidin-4-ylamino)-5-methoxyphenyl)methanesulfonamide

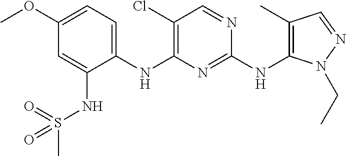

¹H NMR (MeOD) δ 8.27 (s, 1H), 8.09 (br s, 1H), 7.94 (s, 1H), 7.56 (d, 1H), 7.32 (s, 1H), 6.94 (br s, 1H), 6.78 (d, 1H), 3.90 (q, 2H), 3.81 (s, 3H), 2.94 (s, 3H), 1.85 (s, 3H), 1.22 (t, 3H); LCMS method B, (ES+) 452, RT=8.12 min.

### Example 27

### N-(2-(2-(1-sec-butyl-3-methyl-1H-pyrazol-5-ylamino)-5-chloropyrimidin-4-ylamino)-5-methoxyphenyl)methanesulfonamide

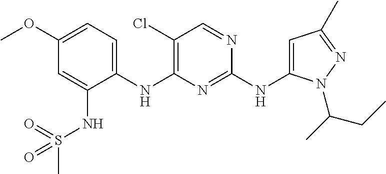

¹H NMR (MeOD) δ 8.08 (br s, 1H), 7.97 (s, 1H), 7.53 (d, 1H), 6.99 (d, 1H), 6.81 (dd, 1H), 5.85 (s, 1H), 4.14-4.06 (m, 1H), 3.82 (s, 3H), 2.91 (s, 3H), 2.20 (s, 3H), 1.83-1.72 (m, 1H), 1.68-1.58 (m, 1H), 1.24 (d, 3H), 0.65 (t, 3H); LCMS method B, (ES+) 481, RT=8.76 min.

### Example 28

### N-(2-(5-chloro-2-(1-ethyl-3-methyl-1H-pyrazol-4-ylamino)pyrimidin-4-ylamino)-5-methoxyphenyl)methanesulfonamide

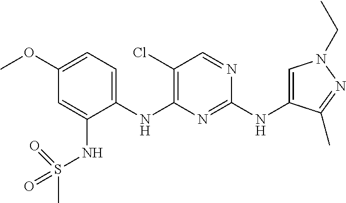

¹H NMR (d₆-DMSO) δ 8.61-8.48 (m, 3H), 8.26 (s, 2H), 8.00 (s, 1H), 6.99 (br s, 1H), 6.72 (br s, 1H), 3.79 (br s, 2H), 3.75 (s, 3H), 2.81 (s, 3H), 2.06 (s, 3H), 1.21 (br s, 3H); LCMS method B, (ES+) 451, RT=6.61 min.

### Example 29

### N-(2-(5-chloro-2-(1-ethyl-1H-pyrazol-5-ylamino)pyrimidin-4-ylamino)-5-methoxyphenyl)methanesulfonamide

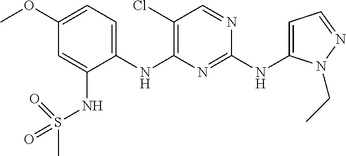

¹H NMR (MeOD) δ 8.25 (s, 1H), 7.99 (s, 1H), 7.56 (d, 1H), 7.39 (d, 1H), 6.98 (d, 1H), 6.82 (dd, 1H), 6.08 (d, 1H), 3.96 (q, 2H), 3.82 (s, 3H), 2.92 (s, 3H), 1.26 (t, 3H); LCMS method B, (ES+) 438, RT=8.00 min.

### Example 30

### N-(2-(2-(1,3-dimethyl-1H-pyrazol-4-ylamino)-5-fluoropyrimidin-4-ylamino)phenyl)methanesulfonamide

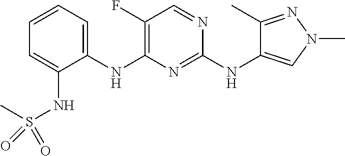

¹H NMR: (d₆-DMSO) δ 10.83 (br s, 1H), 10.51 (br s, 1H) 9.42 (s, 1H), 8.47 (br s, 1H), 7.62 (m, 2H), 7.48 (m, 2H), 7.34 (br s, 1H), 6.83 (s, 1H), 3.48 (s, 3H), 2.94 (s, 3H), 2.09 (s, 3H); LCMS method A, (ES+) 392, RT=1.61 min.

### Example 31

### N-(2-(5-chloro-2-(4-methyl-1,2,5-oxadiazol-3-ylamino)pyrimidin-4-ylamino)-5-methoxyphenyl)methanesulfonamide

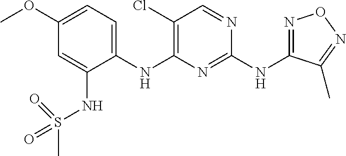

¹H NMR (MeOD) δ 8.07 (s, 1H), 7.67 (d, 1H), 7.01 (d, 1H), 6.84 (dd, 1H), 3.83 (s, 3H), 2.95 (s, 3H), 2.14 (s, 3H); LCMS method B, (ES+) 426, RT=8.95 min.

### Example 32

### N-(2-(2-(1,3-dimethyl-M-pyrazol-4-ylamino)-5-fluoropyrimidin-4-ylamino)-5-methoxyphenyl)methanesulfonamide

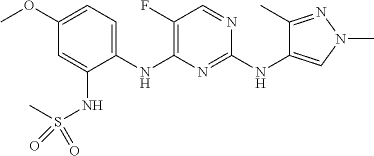

¹H NMR: (d₆-DMSO) δ 8.52 (s, 1H), 8.34 (br s, 1H), 8.14 (s, 1H), 7.96 (d, 1H), 7.43 (d, 1H), 7.24 (br s, 1H), 7.06 (d, 1H), 6.88 (dd, 1H), 3.80 (s, 3H), 3.45 (s, 3H), 2.87 (s, 3H), 2.02 (s, 3H); LCMS method A, (ES+) 422, RT=1.66 min.

### Example 33

### N-(2-(5-chloro-2-(1-ethyl-3-methyl-M-pyrazol-4-ylamino)pyrimidin-4-ylamino)phenyl)methanesulfonamide

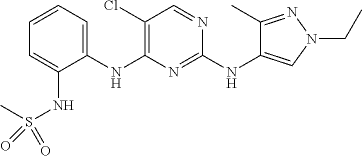

¹H NMR: (d₆-DMSO) δ 9.30 (br s, 1H), 8.65 (br s, 1H), 8.49 (s, 1H), 8.09 (s, 1H), 7.73 (br s, 1H), 7.42 (d, 1H), 7.29 (m, 3H), 3.83 (br s, 2H), 2.92 (s, 3H), 2.06 (s, 3H), 1.23 (br s, 3H); LCMS method A, (ES+) 422, RT=1.93 min.

### Example 34

### N-(2-(2-(1-ethyl-3-methyl-1H-pyrazol-4-ylamino)-5-fluoropyrimidin-4-ylamino)-5-methoxyphenyl)methanesulfonamide

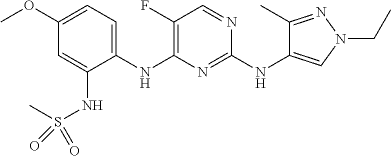

¹H NMR: (d₆-DMSO) δ 9.17 (s, 1H), 8.52 (s, 1H), 8.33 (s, 1H), 7.98 (d, 1H), 7.41 (d, 1H), 7.22 (br s, 1H), 7.06 (d, 1H), 6.86 (m, 1H), 3.78 (m, 5H), 2.87 (s, 3H), 2.05 (s, 3H), 1.19 (t, 3H); LCMS method A, (ES+) 436, RT=1.74 min.

### Example 35

### N-(2-(5-chloro-2-(1,3-dimethyl-1H-pyrazol-4-ylamino)pyrimidin-4-ylamino)phenyl)methanesulfonamide

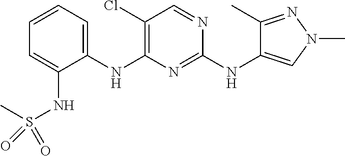

¹H NMR: (d₆-DMSO) δ 10.57 (s, 1H), 10.26 (s, 1H), 9.33 (s, 1H), 8.51 (s, 1H), 7.71-7.93 (m, 4H), 6.73 (s, 1H), 3.47 (s, 3H), 2.93 (s, 3H), 2.10 (s, 3H); LCMS method A, (ES+) 408, RT=1.82 min.

### Example 36

### N-(2-(5-fluoro-2-(isoxazol-3-ylamino)pyrimidin-4-ylamino)-5-methoxyphenyl)methanesulfonamide

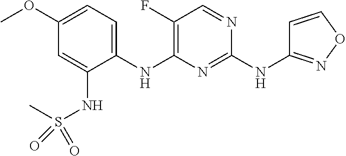

¹H NMR: (MeOD) δ 8.21 (d, 1H), 7.87 (d, 1H), 7.41 (d, 1H), 7.03 (d, 1H), 6.80 (d, 1H), 6.50 (d, 1H), 3.74 (s, 3H), 2.82 (s, 3H); LCMS method A, (ES+) 395, RT=2.10 min.

### Example 37

### N-(2-(2-(1-ethyl-3-methyl-1H-pyrazol-4-ylamino)-5-fluoropyrimidin-4-ylamino)phenyl)methanesulfonamide

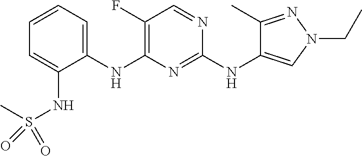

¹H NMR: (d₆-DMSO) δ 10.82 (s, 1H), 10.52 (s, 1H), 9.42 (s, 1H), 8.47 (s, 1H), 7.62 (d, 1H), 7.45 (m, 2H), 7.33 (br s, 1H), 6.88 (s, 1H), 3.74 (br s, 2H), 2.94 (s, 3H), 2.11 (s, 3H), 1.14 (s, 3H); LCMS method A, (ES+) 406, RT=1.70 min.

### Example 38

### N-(2-(5-chloro-2-(isoxazol-3-ylamino)pyrimidin-4-ylamino)phenyl)methanesulfonamide

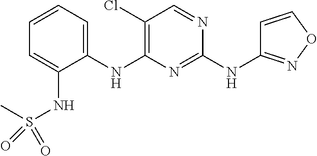

¹H NMR: (d₆-DMSO) δ 10.31 (s, 1H), 9.35 (br s, 1H), 8.69 (s, 1H), 8.59 (s, 1H), 8.22 (s, 1H), 7.98 (d, 1H), 7.44 (dd, 1H), 7.28 (m, 2H), 6.57 (s, 1H), 2.95 (s, 3H); LCMS method A, (ES+) 381, RT=2.28 min.

### Example 39

### N-(2-(5-chloro-2-(1,5-dimethyl-1H-pyrazol-3-ylamino)pyrimidin-4-ylamino)phenyl)methanesulfonamide

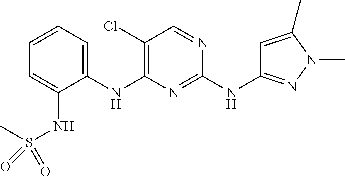

¹H NMR: (d₆-DMSO) δ 10.81 (br s, 1H), 9.97 (s, 1H), 9.34 (s, 1H), 8.40 (s, 1H), 7.54 (d, 2H), 7.42 (m, 1H), 7.34 (m, 1H), 5.60 (br s, 1H), 3.65 (s, 3H), 2.96 (s, 3H), 2.12 (s, 3H); LCMS method A, (ES+) 408, RT=1.94 min.

### Example 40

### N-(2-(5-chloro-2-(1-isopropyl-1H-pyrazol-3-ylamino)pyrimidin-4-ylamino)phenyl)methanesulfonamide

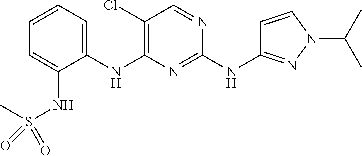

¹H NMR: (d₆-DMSO) δ 10.94 (br s, 1H), 9.98 (s, 1H), 9.35 (s, 1H), 7.58 (br s, 1H), 7.54 (d, 1H), 7.40 (t, 1H), 7.33 (t, 1H), 5.78 (br s, 1H), 4.14 (br s, 1H), 2.96 (s, 3H), 1.37 (s, 6H); LCMS method A, (ES+) 422, RT=2.12 min.

### Example 41

### N-(2-(5-bromo-2-(1-methyl-1H-pyrazol-4-ylamino)pyrimidin-4-ylamino)phenyl)methanesulfonamide

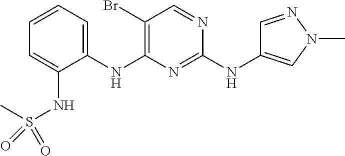

¹H NMR: (d₆-DMSO) δ 10.68 (s, 1H), 9.80 (s, 1H), 9.31 (s, 1H), 7.57 (m, 1H), 7.49 (m, 2H), 7.39 (m, 1H), 7.18 (s, 1H), 6.99 (s, 1H), 3.59 (s, 3H), 2.94 (s, 3H); LCMS method A, (ES+) 440, RT=1.85 min.

### Example 42

### N-(2-(5-chloro-2-(1-ethyl-1H-pyrazol-3-ylamino)pyrimidin-4-ylamino)phenyl)methanesulfonamide

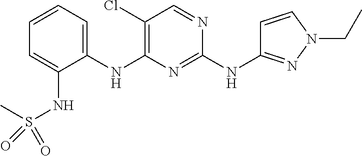

¹H NMR: (d₆-DMSO) δ 9.62 (s, 1H), 9.30 (s, 1H), 8.52 (s, 1H), 8.14 (s, 1H), 8.09 (br s, 1H), 7.45 (d, 1H), 7.40 (dd, 1H), 7.31 (td, 1H), 7.22 (td, 1H), 6.14 (br s, 1H), 3.98 (q, 2H), 2.95 (s, 3H), 1.32 (t, 3H); LCMS method A, (ES+) 408, RT=2.00 min.

Examples 43-101 in Table 4 can be synthesised by the procedures outlined in Examples 1 and 2, using the appropriate reactants.

TABLE 4

| | | Analytical Data | | |
|---|---|---|---|---|
| | | LCMS | | |
| Example | Chemical Name | Method | RT | ES+ |
| 43 | N-(2-((5-bromo-2-((1-methyl-1H-pyrazol-3- | A | 1.9 | 440 |
| | yl)amino)pyrimidin-4- | | | |
| | yl)amino)phenyl)methanesulfonamide | | | |
| 44 | N-(2-((5-chloro-2-(isoxazol-4-ylamino)pyrimidin-4- | A | 2.25 | 381 |
| | yl)amino)phenyl)methanesulfonamide | | | |
| 45 | N-(2-((5-chloro-2-((3-methylisoxazol-5- | A | 1.58 | 395 |
| | yl)amino)pyrimidin-4- | | | |
| | yl)amino)phenyl)methanesulfonamide | | | |
| 46 | N-(2-((5-chloro-2-((1-propyl-1H-pyrazol-3- | A | 2.16 | 422 |
| | yl)amino)pyrimidin-4- | | | |
| | yl)amino)phenyl)methanesulfonamide | | | |
| 47 | N-(2-((2-((1,5-dimethyl-1H-pyrazol-3-yl)amino)-5- | A | 1.78 | 422 |
| | fluoropyrimidin-4-yl)amino)-5- | | | |
| | methoxyphenyl)methanesulfonamide | | | |
| 48 | N-(2-((5-fluoro-2-((1-isopropyl-1H-pyrazol-3- | A | 1.9 | 436 |
| | yl)amino)pyrimidin-4-yl)amino)-5- | | | |
| | methoxyphenyl)methanesulfonamide | | | |
| 49 | N-(2-((5-bromo-2-((1-isopropyl-1H-pyrazol-3- | A | 2.14 | 468 |
| | yl)amino)pyrimidin-4- | | | |
| | yl)amino)phenyl)methanesulfonamide | | | |
| 50 | N-(2-((5-bromo-2-(isoxazol-3-ylamino)pyrimidin-4- | A | 2.29 | 427 |
| | yl)amino)phenyl)methanesulfonamide | | | |
| 51 | N-(2-((5-bromo-2-((1,5-dimethyl-1H-pyrazol-3- | A | 1.97 | 452 |
| | yl)amino)pyrimidin-4- | | | |
| | yl)amino)phenyl)methanesulfonamide | | | |
| 52 | N-(2-((5-chloro-2-((1-(2-methoxyethyl)-1H-pyrazol-4- | A | 1.89 | 438 |
| | yl)amino)pyrimidin-4- | | | |
| | yl)amino)phenyl)methanesulfonamide | | | |
| 53 | N-(2-((5-chloro-2-((1-methyl-1H-pyrazol-3- | B | 7.18 | 420 |
| | yl)amino)pyrimidin-4- | | | |
| | yl)amino)phenyl)cyclopropanesulfonamide | | | |
| 54 | N-(2-((5-chloro-2-((1-isopropyl-1H-pyrazol-3- | B | 8.23 | 448 |
| | yl)amino)pyrimidin-4- | | | |
| | yl)amino)phenyl)cyclopropanesulfonamide | | | |
| 55 | N-(2-((5-chloro-2-((1,5-dimethyl-1H-pyrazol-3- | B | 7.39 | 434 |
| | yl)amino)pyrimidin-4- | | | |
| | yl)amino)phenyl)cyclopropanesulfonamide | | | |
| 56 | N-(2-((5-chloro-2-((1-methyl-1H-pyrazol-3- | A | 1.96 | 408 |
| | yl)amino)pyrimidin-4-yl)amino)phenyl)ethanesulfonamide | | | |
| 57 | N-(2-((5-chloro-2-((1-methyl-1H-pyrazol-4- | A | 1.91 | 408 |
| | yl)amino)pyrimidin-4-yl)amino)phenyl)ethanesulfonamide | | | |
| 58 | N-(2-((5-chloro-2-((1-methyl-1H-pyrazol-4- | A | 2 | 420 |
| | yl)amino)pyrimidin-4- | | | |
| | yl)amino)phenyl)cyclopropanesulfonamide | | | |
| 59 | N-(2-((5-chloro-2-((1-ethyl-5-methyl-1H-pyrazol-3- | A | 2.08 | 422 |
| | yl)amino)pyrimidin-4- | | | |
| | yl)amino)phenyl)methanesulfonamide | | | |
| 60 | N-(2-((5-chloro-2-((1-(cyclopropylmethyl)-1H-pyrazol-5- | A | 2.35 | 464 |
| | yl)amino)pyrimidin-4-yl)amino)-5- | | | |
| | methoxyphenyl)methanesulfonamide | | | |
| 61 | N-(2-((2-((1,5-dimethyl-1H-pyrazol-3-yl)amino)-5- | B | 6.33 | 406 |
| | fluoropyrimidin-4-yl)amino)phenyl)ethanesulfonamide | | | |
| 62 | N-(2-((5-fluoro-2-((1-methyl-1H-pyrazol-4- | B | 5.61 | 392 |
| | yl)amino)pyrimidin-4-yl)amino)phenyl)ethanesulfonamide | | | |
| 63 | N-(2-((5-fluoro-2-((1-methyl-1H-pyrazol-3- | B | 5.94 | 392 |
| | yl)amino)pyrimidin-4-yl)amino)phenyl)ethanesulfonamide | | | |
| 64 | N-(2-((5-fluoro-2-((1-(2-methoxyethyl)-1H-pyrazol-3- | B | 6 | 436 |
| | yl)amino)pyrimidin-4-yl)amino)phenyl)ethanesulfonamide | | | |
| 65 | N-(2-((5-chloro-2-((1-(3-cyanopropyl)-1H-pyrazol-4- | A | 1.92 | 447 |
| | yl)amino)pyrimidin-4- | | | |
| | yl)amino)phenyl)methanesulfonamide | | | |
| 66 | N-(2-((2-((1-(3-cyanopropyl)-1H-pyrazol-4-yl)amino)-5- | A | 1.68 | 431 |
| | fluoropyrimidin-4-yl)amino)phenyl)methanesulfonamide | | | |
| 67 | N-(2-((5-chloro-2-((1,5-dimethyl-1H-pyrazol-4- | A | 1.75 | 438 |
| | yl)amino)pyrimidin-4-yl)amino)-5- | | | |
| | methoxyphenyl)methanesulfonamide | | | |
| 68 | N-(2-((5-chloro-2-((1-isopropyl-1H-pyrazol-5- | A | 2.28 | 452 |
| | yl)amino)pyrimidin-4-yl)amino)-5- | | | |
| | methoxyphenyl)methanesulfonamide | | | |
| 69 | N-(2-((5-chloro-2-((3-methyl-1-propyl-1H-pyrazol-5- | A | 2.28 | 466 |
| | yl)amino)pyrimidin-4-yl)amino)-5- | | | |
| | methoxyphenyl)methanesulfonamide | | | |
| 70 | N-(2-((5-chloro-2-((1-methyl-1H-pyrazol-3- | B | 6.75 | 412 |
| | yl)amino)pyrimidin-4-yl)amino)-6- | | | |
| | fluorophenyl)methanesulfonamide | | | |
| 71 | N-(2-((5-chloro-2-((1-isopropyl-3-methyl-1H-pyrazol-5- | B | 7.9 | 467 |
| | yl)amino)pyrimidin-4-yl)amino)-5- | | | |
| | methoxyphenyl)methanesulfonamide | | | |
| 72 | N-(2-((2-((1-(2,2-difluoroethyl)-3-methyl-1H-pyrazol-4- | C | 2.05 | 442 |
| | yl)amino)-5-fluoropyrimidin-4- | | | |
| | yl)amino)phenyl)methanesulfonamide | | | |
| 73 | N-(2-((2-((1-(2,2-difluoroethyl)-3-methoxy-1H-pyrazol-4- | C | 2.07 | 458 |
| | yl)amino)-5-fluoropyrimidin-4- | | | |
| | yl)amino)phenyl)methanesulfonamide | | | |
| 74 | N-(2-((5-fluoro-2-((3-methoxy-1-(2,2,2-trifluoroethyl)-1H- | C | 2.13 | 476 |
| | pyrazol-4-yl)amino)pyrimidin-4- | | | |
| | yl)amino)phenyl)methanesulfonamide | | | |
| 75 | N-(2-((5-fluoro-2-((5-methyl-1-(2,2,2-trifluoroethyl)-1H- | C | 2.12 | 460 |
| | pyrazol-3-yl)amino)pyrimidin-4- | | | |
| | yl)amino)phenyl)methanesulfonamide | | | |
| 76 | N-(2-((5-chloro-2-((1-propyl-1H-pyrazol-5- | A | 2.32 | 452 |
| | yl)amino)pyrimidin-4-yl)amino)-5- | | | |
| | methoxyphenyl)methanesulfonamide | | | |
| 77 | N-(2-((5-chloro-2-((1-ethyl-5-methyl-1H-pyrazol-4- | A | 1.84 | 452 |
| | yl)amino)pyrimidin-4-yl)amino)-5- | | | |
| | methoxyphenyl)methanesulfonamide | | | |
| 78 | N-(2-((5-chloro-2-((1,5-dimethyl-1H-pyrazol-3- | A | 2.08 | 426 |
| | yl)amino)pyrimidin-4-yl)amino)-6- | | | |
| | fluorophenyl)methanesulfonamide | | | |
| 79 | N-(2-((5-chloro-2-((1-propyl-1H-pyrazol-3- | A | 2.29 | 440 |
| | yl)amino)pyrimidin-4-yl)amino)-6- | | | |
| | fluorophenyl)methanesulfonamide | | | |
| 80 | N-(2-((5-chloro-2-((1-(2,2-difluoroethyl)-3-methoxy-1H- | C | 2.09 | 474 |
| | pyrazol-4-yl)amino)pyrimidin-4- | | | |
| | yl)amino)phenyl)methanesulfonamide | | | |
| 81 | N-(2-((5-chloro-2-((1-(2,2-difluoroethyl)-3-methyl-1H- | C | 2 | 458 |
| | pyrazol-4-yl)amino)pyrimidin-4- | | | |
| | yl)amino)phenyl)methanesulfonamide | | | |
| 82 | N-(2-((5-chloro-2-((3-methoxy-1-(2,2,2-trifluoroethyl)-1H- | C | 2.12 | 492 |
| | pyrazol-4-yl)amino)pyrimidin-4- | | | |
| | yl)amino)phenyl)methanesulfonamide | | | |
| 83 | N-(2-((5-chloro-2-((5-methyl-1-(2,2,2-trifluoroethyl)-1H- | C | 2.07 | 476 |
| | pyrazol-3-yl)amino)pyrimidin-4- | | | |
| | yl)amino)phenyl)methanesulfonamide | | | |
| 84 | N-(2-((5-chloro-2-((1-(2,2-difluoroethyl)-1H-pyrazol-4- | C | 2.01 | 434 |
| | yl)amino)pyrimidin-4- | | | |
| | yl)amino)phenyl)methanesulfonamide | | | |
| 85 | N-(2-((2-((1-(2,2-difluoroethyl)-1H-pyrazol-4-yl)amino)-5- | C | 2.04 | 428 |
| | fluoropyrimidin-4-yl)amino)phenyl)methanesulfonamide | | | |
| 86 | N-(2-((2-((1-butyl-3-methyl-1H-pyrazol-5-yl)amino)-5- | A | 2.45 | 480 |
| | chloropyrimidin-4-yl)amino)-5- | | | |
| | methoxyphenyl)methanesulfonamide | | | |
| 87 | N-(2-((2-((1-butyl-1H-pyrazol-5-yl)amino)-5- | A | 2.48 | 466 |
| | chloropyrimidin-4-yl)amino)-5- | | | |
| | methoxyphenyl)methanesulfonamide | | | |
| 88 | N-(2-((5-chloro-2-((1-(1-cyclopropylethyl)-3-methyl-1H- | A | 2.44 | 492 |
| | pyrazol-5-yl)amino)pyrimidin-4-yl)amino)-5- | | | |
| | methoxyphenyl)methanesulfonamide | | | |
| 89 | N-(2-((5-chloro-2-((1-methyl-1H-pyrazol-4- | A | 1.98 | 412 |
| | yl)amino)pyrimidin-4-yl)amino)-6- | | | |
| | fluorophenyl)methanesulfonamide | | | |
| 90 | N-(2-((5-chloro-2-((1-ethyl-1H-pyrazol-3- | A | 2.61 | 504 |
| | yl)amino)pyrimidin-4-yl)amino)-6- | | | |
| | fluorophenyl)methanesulfonamide | | | |
| 91 | N-(2-((5-chloro-2-((1-ethyl-5-methyl-1H-pyrazol-3- | A | 2.21 | 440 |
| | yl)amino)pyrimidin-4-yl)amino)-6- | | | |
| | fluorophenyl)methanesulfonamide | | | |
| 92 | 3-((5-chloro-4-((2- | A | 1.97 | 438 |
| | (methylsulfonamido)phenyl)amino)pyrimidin-2-yl)amino)- | | | |
| | 1-methyl-1H-pyrazole-5-carboxylic acid | | | |
| 93 | N-(2-((5-chloro-2-((1-(2-methoxyethyl)-1H-pyrazol-4- | A | 2.1 | 456 |
| | yl)amino)pyrimidin-4-yl)amino)-6- | | | |
| | fluorophenyl)methanesulfonamide | | | |
| 94 | N-(2-((5-chloro-2-((1-(2-hydroxyethyl)-3-methyl-1H- | A | 2.07 | 468 |
| | pyrazol-5-yl)amino)pyrimidin-4-yl)amino)-5- | | | |
| | methoxyphenyl)methanesulfonamide | | | |
| 95 | N-(2-((5-chloro-2-((1-isobutyl-3-methyl-1H-pyrazol-5- | A | 2.44 | 480 |
| | yl)amino)pyrimidin-4-yl)amino)-5- | | | |
| | methoxyphenyl)methanesulfonamide | | | |
| 96 | N-(2-((5-chloro-2-((1-(2-methoxyethyl)-3-methyl-1H- | B | 7.81 | 482 |
| | pyrazol-5-yl)amino)pyrimidin-4-yl)amino)-5- | | | |
| | methoxyphenyl)methanesulfonamide | | | |
| 97 | 3-((5-chloro-4-((2- | A | 1.9 | 451 |
| | (methylsulfonamido)phenyl)amino)pyrimidin-2-yl)amino)- | | | |
| | N,1-dimethyl-1H-pyrazole-5-carboxamide | | | |
| 98 | N-(2-((5-chloro-2-((1-isobutyl-1H-pyrazol-5- | A | 2.55 | 466 |
| | yl)amino)pyrimidin-4-yl)amino)-5- | | | |
| | methoxyphenyl)methanesulfonamide | | | |
| 99 | N-(2-fluoro-6-((5-fluoro-2-((1-methyl-1H-pyrazol-4- | A | 1.72 | 396 |
| | yl)amino)pyrimidin-4- | | | |
| | yl)amino)phenyl)methanesulfonamide | | | |
| 100 | 3-((5-chloro-4-((2-(methylsulfonamido)phenyl)amino) | A | 2.02 | 465 |
| | pyrimidin-2-yl)amino)-N-ethyl-1-methyl-1H-pyrazole-5- | | | |
| | carboxamide | | | |
| 101 | 3-((5-chloro-4-((2-(methylsulfonamido)phenyl)amino) | A | 1.79 | 480 |
| | pyrimidin-2-yl)amino)-N-(2-hydroxyethyl)-1-methyl-1H- | | | |
| | pyrazole-5-carboxamide | | | |

### Example 102

### Determination of the Effect of the Compounds According to the Invention on ZAP-70

The compounds of the present invention as described in the previous examples were tested in the ZAP-70 kinobeads assay as described (EP-A 1862802 and WO-A 2007/137867). Briefly, test compounds (at various concentrations) and the affinity matrix with the immobilized aminopyrido-pyrimidine ligand 24 are added to cell lysate aliquots and allowed to bind to the proteins in the lysate sample. After the incubation time the beads with captured proteins are separated from the lysate. Bound proteins are then eluted and the presence ZAP-70 is detected and quantified using a specific antibody in a dot blot procedure and the Odyssey infrared detection system.

In general, compounds of the invention are effective for the inhibition of ZAP-70, with an IC₅₀ of ≦10 μM.

By this method (ZAP-70 kinobeads assay) the following compounds demonstrated an IC₅₀ value of 1 μM<IC₅₀≦10 μM: Examples 1, 14, 15, 17, 19, 26, 31, 36, 37, 38, 44, 88, 98.

In addition, the following compounds demonstrated an IC₅₀ between 0.1 μM<IC_{50≦1} μM: Examples 3, 4, 7, 11, 13, 16, 20, 21, 22, 23, 24, 27, 28, 29, 30, 32, 33, 34, 39, 40, 41, 42, 43, 60, 68, 71, 76, 87, 94, 95, 96.

In addition, the following compounds demonstrated an IC₅₀≦0.1 μM: Examples 2, 5, 6, 8, 12, 18, 35, 67, 69, 77, 86.

### Example 103

### Determination of the Effect of the Compounds According to the Invention on JAK3

Compounds were tested by the procedure described in Example 102 but using a JAK3 specific antibody (Cell Signaling Technology, Inc., Danvers, US, catalogue number 3775) instead of a ZAP70 antibody.

By this method (JAK3 kinobeads assay) the following compounds demonstrated an IC₅₀ value of 1 μM<IC₅₀≦10 μM: Examples 10, 13, 17, 29, 31, 54, 61, 63, 74, 75, 94, 96.

In addition, the following compounds demonstrated an IC₅₀ of 0.1 μM<IC₅₀≦1 μM: Examples 1, 5, 7, 9, 11, 12, 30, 32, 34, 36, 37, 44, 47, 48, 53, 55, 56, 62, 4, 67, 72, 73, 77, 78, 79, 82, 83.

In addition, the following compounds demonstrated an IC₅₀≦0.1 μM: Examples 2, 3, 4, 6, 8, 16, 18, 20, 21, 22, 23, 24, 25, 28, 33, 35, 38, 39, 40, 41, 42, 43, 46, 49, 50, 51, 52, 57, 58, 59, 65, 66, 70, 80, 81, 84, 85, 89, 90, 91, 92, 93, 97, 99, 100, 101.

1. A compound of formula (I) 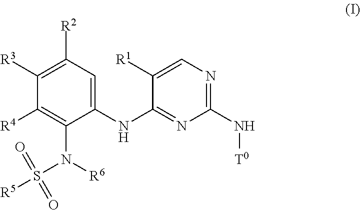
or a pharmaceutically acceptable salt, tautomer, prodrug or metabolite thereof, wherein
T⁰ is 5 membered aromatic heterocyclyl, wherein T⁰ is optionally substituted with one or more R⁷, which are the same or different;
R⁷ is halogen; CN; C(O)OR⁸; OR⁸; C(O)R⁸; C(O)N(R⁸R^{8a}); S(O)₂N(R⁸R^{8a}); S(O)N(R⁸R^{8a}); S(O)₂R⁸; S(O)R⁸; N(R⁸)S(O)₂N(R^{8a}R^{8b}); SR⁸; N(R⁸R^{8a}); NO₂; OC(O)R⁸; N(R⁸)C(O)R^{8a}; N(R⁸)S(O)₂R^{8a}; N(R⁸)S(O)R^{8a}; N(R⁸)C(O)N(R^{8a}R^{8b}); N(R⁸)C(O)OR^{8a}; OC(O)N(R⁸R^{8a}); C₁₋₆ alkyl; C₂₋₆ alkenyl; C₂₋₆ alkynyl; or T¹, wherein C₁₋₆ alkyl; C₂₋₆ alkenyl; and C₂₋₆ alkynyl are optionally substituted with one or more R⁹, which are the same or different;
R⁸, R^{8a}, R^{8b} are independently selected from the group consisting of H; T¹; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁰, which are the same or different;
R⁹, R¹⁰ are independently selected from the group consisting of T¹; halogen; CN; C(O)OR¹¹; OR¹¹; C(O)R¹¹; C(O)N(R¹¹R^{11a}); S(O)₂N(R¹¹R^{11a}); S(O)N(R¹¹R^{11a}); S(O)₂R¹¹; S(O)R¹¹; N(R¹¹)S(O)₂N(R^{11a}R^{11b}); N(R¹¹)S(O)N(R^{11a}R^{11b}); SR¹¹; N(R¹¹R^{11a}); NO₂; OC(O)R¹¹; N(R¹¹)C(O)R^{11a}; N(R¹¹)S(O)₂R^{11a}; N(R¹¹)S(O)R^{11a}; N(R¹¹)C(O)N(R^{11a}R^{11b}); N(R¹¹)C(O)OR^{11a}; and OC(O)N(R¹¹R^{11a});
R¹¹; R^{11a}; R^{11b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T¹ is phenyl; C₃₋₇ cycloalkyl; or 4 to 7 membered heterocyclyl, wherein T¹ is optionally substituted with one or more R¹², which are the same or different;
R¹² is halogen; CN; C(O)OR¹³; OR¹³; oxo (═O), where the ring is at least partially saturated; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); N(R¹³)S(O)N(R^{13a}R^{13b}); SR¹³; N(R¹³R^{13a}); NO₂; OC(O)R¹³; N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R¹⁴, which are the same or different;
R¹³; R^{13a}, R^{13b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁵, which are the same or different;
R¹⁴, R¹⁵ are independently selected from the group consisting of halogen; CN; C(O)OR¹⁶; OR¹⁶; C(O)R¹⁶; C(O)N(R¹⁶R^{16a}); S(O)₂N(R¹⁶R^{16a}); S(O)N(R¹⁶R^{16a}); S(O)₂R¹⁶; S(O)R¹⁶; N(R¹⁶)S(O)₂N(R^{16a}R^{16b}); N(R¹⁶)S(O)N(R^{16a}R^{16b}); SR¹⁶; N(R¹⁶R^{16a}); NO₂; OC(O)R¹⁶;
N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; and OC(O)N(R¹⁶R^{16a});
R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R¹ is H; F; Cl; Br; CN; C₁₋₄ alkyl; CH₂F; CHF₂; CF₃; OH; OCH₃; NO₂; NH₂; NHCH₃; N(CH₃)₂; or NO₂;
R², R³, R⁴ are independently selected from the group consisting of H; halogen; CN; C(O)OR¹⁷; OR¹⁷; C(O)R¹⁷; C(O)N(R¹⁷R^{17a}); S(O)₂N(R¹⁷R^{17a}); S(O)N(R¹⁷R^{17a}); S(O)₂R¹⁷; S(O)R¹⁷; SR¹⁷; N(R¹⁷R^{17a}); NO₂; OC(O)R¹⁷; N(R¹⁷)C(O)R^{17a}; N(R¹⁷)S(O)₂R^{17a}; N(R¹⁷)S(O)R^{17a}; N(R¹⁷)C(O)N(R^{17a}R^{17b}); N(R¹⁷)C(O)OR^{17a}; OC(O)N(R¹⁷R^{17a}); C₁₋₆ alkyl; C₂₋₆ alkenyl; C₂₋₆ alkynyl; and T², wherein C₁₋₆ alkyl; C₂₋₆ alkenyl; and C₂₋₆ alkynyl are optionally substituted with one or more R¹⁸, which are the same or different;
Optionally, one of the pairs R²/R³, R³/R⁴ is joined together with the phenyl ring to which it is attached to form a bicyclic ring T³;
R¹⁷, R^{17a}, R^{17b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁹, which are the same or different;
R¹⁸, R¹⁹ are independently selected from the group consisting of T²; halogen; CN; C(O)OR²⁰; OR²⁰; C(O)R²⁰; C(O)N(R²⁰R^{20a}); S(O)₂N(R²⁰R^{20a}); S(O)N(R²⁰R^{20a}); S(O)₂R²⁰; S(O)R²⁰); N(R²⁰)S(O)₂N(R^{20a}R^{20b}); N(R²⁰)S(O)N(R^{20a}R^{20b}); SR²⁰; N(R²⁰R^{20a}); NO₂; OC(O)R²⁰); N(R²⁰)C(O)R^{20a}); N(R²⁰)S(O)₂R^{20a}); N(R²⁰)S(O)R^{20a}); N(R²⁰)C(O)N(R^{20a}R^{20b}); N(R²⁰)C(O)OR^{20a}; and OC(O)N(R²⁰R^{20a});
R²⁰, R^{20a}, R^{20b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T² is phenyl; C₃₋₇ cycloalkyl; or 4 to 7 membered heterocyclyl, wherein T² is optionally substituted with one or more R²¹, which are the same or different;
T³ is naphthyl; indenyl; indanyl; or 9 to 11 membered benzo-fused heterobicyclyl, wherein T³ is optionally substituted with one or more R²², which are the same or different;
R²¹, R²² are independently selected from the group consisting of halogen; CN; C(O)OR²³; OR²³; oxo (═O), where the ring is at least partially saturated; C(O)R²³; C(O)N(R²³R^{23a}); S(O)₂N(R²³R^{23a}); S(O)N(R²³R^{23a}); S(O)₂R²³; S(O)R²³; N(R²³)S(O)₂N(R^{23a}R^{23b}); N(R²³)S(O)N(R^{23a}R^{23b}); SR²³; N(R²³R^{23a}); NO₂; OC(O)R²³; N(R²³)C(O)R^{23a}; N(R²³)S(O)₂R^{23a}; N(R²³)S(O)R^{23a}; N(R²³)C(O)N(R^{23a}R^{23b}); N(R²³)C(O)OR^{23a}; OC(O)N(R²³R^{23a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R²³, R^{23a}, R^{23b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R⁵ is R²⁴; or N(R²⁴R^{24a});
R²⁴ is T4; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R²⁵, which are the same or different;
R^{24a} is H; or C₁₋₄ alkyl, wherein C₁₋₄ alkyl is optionally substituted with one or more F;
R²⁵ is T⁴; halogen; CN; C(O)OR²⁶; OR²⁶; C(O)R²⁶; C(O)N(R²⁶R^{26a}); S(O)₂N(R²⁶R^{26a}); S(O)N(R²⁶R^{26a}); S(O)₂R²⁶; S(O)R²⁶; N(R²⁶)S(O)₂N(R^{26a}R^{26b}); N(R²⁶)S(O)N(R^{26a}R²⁶); SR²⁶; N(R²⁶R^{26a}); NO₂; OC(O)R²⁶; N(R²⁶)C(O)R^{26a}; N(R²⁶)S(O)₂R^{26a}; N(R²⁶)S(O)R^{26a}; N(R²⁶)C(O)N(R^{26a}R^{26b}); N(R²⁶)C(O)OR^{26a}; or OC(O)N(R²⁶R^{26a});
R²⁶, R^{26a}; R^{26b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T⁴ is phenyl; C₃₋₇ cycloalkyl; or 4 to 7 membered heterocyclyl, wherein T⁴ is optionally substituted with one or more R²⁷, which are the same or different;
R²⁷ is halogen; CN; C(O)OR²⁸; OR²⁸; oxo (═O), where the ring is at least partially saturated; C(O)R²⁸; C(O)N(R²⁸R^{28a}); S(O)₂N(R²⁸R^{28a}); S(O)N(R²⁸R^{28a}); S(O)₂R²⁸; S(O)R²⁸; N(R²⁸)S(O)₂N(R^{28a}R²⁸); N(R²⁸)S(O)N(R^{28a}R²⁸); SR²⁸; N(R²⁸R^{28a}); NO₂; OC(O)R²⁸; N(R²⁸)C(O)R^{28a}; N(R²⁸)S(O)₂R^{28a}; N(R²⁸)S(O)R^{28a}; N(R²⁸)C(O)N(R^{28a}R^{28b}); N(R²⁸)C(O)OR^{28a}; OC(O)N(R²⁸R^{28a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;
R²⁸, R^{28a}; R^{28b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R⁶ is H; C₁₋₄ alkyl; C₃₋₅ cycloalkyl; or C₃₋₅ cycloalkylmethyl, wherein C₁₋₄ alkyl; C₃₋₅ cycloalkyl and C₃₋₅ cycloalkylmethyl are optionally substituted with one or more halogen, which are the same or different.

2. A compound of claim 1, wherein T⁰ is a 5 membered aromatic heterocycle which is attached to the amine in formula (I) via a carbon ring atom and wherein T⁰ is unsubstituted or substituted with one, two or three R⁷, which are the same or different.

3. A compound of claim 1, wherein T⁰ is unsubstituted or substituted and selected from the group consisting of pyrazole; isoxazole; and oxadiazole.

4. A compound of claim 1, wherein R⁷ is unsubstituted O—C₁₋₆ alkyl; unsubstituted C₁₋₆ alkyl; or T¹.

5. A compound of claim 1, wherein R¹ is H; F; Cl; Br; CH₃; or CF₃.

6. A compound of claim 1, wherein at least two of R², R³, R⁴ are H.

7. A compound of claim 1, wherein R², R³, R⁴ are independently selected from the group consisting of H; F; Cl; unsubstituted C₁₋₆ alkyl; and unsubstituted O—C₁₋₆ alkyl.

8. A compound of claim 1, wherein R⁵ is R²⁴.

9. A compound of claim 1, wherein R²⁴ is unsubstituted C₁₋₄ alkyl.

10. A compound of claim 1, wherein R⁶ is H.

11. A compound of claim 1 selected from the group consisting of
N-(2-(5-fluoro-2-(1-methyl-1H-pyrazol-3-ylamino)pyrimidin-4-ylamino)phenyl)methanesulfonamide;
N-(2-(5-chloro-2-(3-methyl-1H-pyrazol-4-ylamino)pyrimidin-4-ylamino)-5-methoxyphenyl)methanesulfonamide;
N-(2-(5-chloro-2-(isoxazol-3-ylamino)pyrimidin-4-ylamino)-5-methoxyphenyl)methanesulfonamide;
N-(2-(5-chloro-2-(1-methyl-1H-pyrazol-3-ylamino)pyrimidin-4-ylamino)-5-methoxyphenyl)methanesulfonamide;
N-(2-(5-chloro-2-(1-methyl-1H-pyrazol-5-ylamino)pyrimidin-4-ylamino)-5-methoxyphenyl)methanesulfonamide;
N-(2-(5-chloro-2-(1-methyl-1H-pyrazol-4-ylamino)pyrimidin-4-ylamino)-5-methoxyphenyl)methanesulfonamide;
N-(2-(5-fluoro-2-(1-methyl-1H-pyrazol-3-ylamino)pyrimidin-4-ylamino)-5-methoxyphenyl)methanesulfonamide;
N-(2-(5-chloro-2-(1,3-dimethyl-1H-pyrazol-5-ylamino)pyrimidin-4-ylamino)-5-methoxyphenyl)methanesulfonamide;
N-(2-(5-chloro-2-(3-cyclopropyl-1-methyl-1H-pyrazol-5-ylamino)pyrimidin-4-ylamino)-5-methoxyphenyl)methanesulfonamide;
N-(2-(2-(3-tert-butyl-1-methyl-1H-pyrazol-5-ylamino)-5-chloropyrimidin-4-ylamino)-5-methoxyphenyl)methanesulfonamide;
N-(2-(5-chloro-2-(3-isopropyl-1-methyl-1H-pyrazol-5-ylamino)pyrimidin-4-ylamino)-5-methoxyphenyl)methanesulfonamide;
N-(2-(5-chloro-2-(1-ethyl-3-methyl-1H-pyrazol-5-ylamino)pyrimidin-4-ylamino)-5-methoxyphenyl)methane sulfonamide;
N-(2-(5-chloro-2-(1,3,5-trimethyl-1H-pyrazol-4-ylamino)pyrimidin-4-ylamino)-5-methoxyphenyl)methane sulfonamide;
N-(2-(5-chloro-2-(1,4-dimethyl-1H-pyrazol-5-ylamino)pyrimidin-4-ylamino)-5-methoxyphenyl)methane sulfonamide;
N-(2-(5-chloro-2-(3-ethyl-1,4-dimethyl-1H-pyrazol-5-ylamino)pyrimidin-4-ylamino)-5-methoxyphenyl)methane sulfonamide;
N-(2-(5-chloro-2-(1-methyl-1H-pyrazol-3-ylamino)pyrimidin-4-ylamino)phenyl)methane sulfonamide;
N-(2-(5-chloro-2-(4-methoxy-1,2,5-oxadiazol-3-ylamino)pyrimidin-4-ylamino)-5-methoxyphenyl)methane sulfonamide;
N-(2-(5-chloro-2-(1,3-dimethyl-1H-pyrazol-4-ylamino)pyrimidin-4-ylamino)-5-methoxyphenyl)methane sulfonamide;
N-(2-(5-chloro-2-(3,5-dimethylisoxazol-4-ylamino)pyrimidin-4-ylamino)-5-methoxyphenyl)methane sulfonamide;
N-(2-(5-chloro-2-(1-methyl-1H-pyrazol-4-ylamino)pyrimidin-4-ylamino)phenyl)methane sulfonamide;
N-(2-(5-fluoro-2-(1-methyl-1H-pyrazol-4-ylamino)pyrimidin-4-ylamino)-5-methoxyphenyl)methane sulfonamide;
N-(2-(5-fluoro-2-(1-methyl-1H-pyrazol-4-ylamino)pyrimidin-4-ylamino)phenyl)methane sulfonamide;
N-(2-(2-(1H-pyrazol-4-ylamino)-5-fluoropyrimidin-4-ylamino)phenyl)methane sulfonamide;
N-(2-(2-(1H-pyrazol-4-ylamino)-5-fluoropyrimidin-4-ylamino)-5-methoxyphenyl)methane sulfonamide;
N-(2-(2-(1H-pyrazol-4-ylamino)-5-chloropyrimidin-4-ylamino)phenyl)methane sulfonamide;
N-(2-(5-chloro-2-(1-ethyl-4-methyl-1H-pyrazol-5-ylamino)pyrimidin-4-ylamino)-5-methoxyphenyl)methanesulfonamide;
N-(2-(2-(1-sec-butyl-3-methyl-1H-pyrazol-5-ylamino)-5-chloropyrimidin-4-ylamino)-5-methoxyphenyl)methanesulfonamide;
N-(2-(5-chloro-2-(1-ethyl-3-methyl-1H-pyrazol-4-ylamino)pyrimidin-4-ylamino)-5-methoxyphenyl)methanesulfonamide;
N-(2-(5-chloro-2-(1-ethyl-1H-pyrazol-5-ylamino)pyrimidin-4-ylamino)-5-methoxyphenyl)methanesulfonamide;
N-(2-(2-(1,3-dimethyl-1H-pyrazol-4-ylamino)-5-fluoropyrimidin-4-ylamino)phenyl)methanesulfonamide;
N-(2-(5-chloro-2-(4-methyl-1,2,5-oxadiazol-3-ylamino)pyrimidin-4-ylamino)-5-methoxyphenyl)methanesulfonamide;
N-(2-(2-(1,3-dimethyl-1H-pyrazol-4-ylamino)-5-fluoropyrimidin-4-ylamino)-5-methoxyphenyl)methanesulfonamide;
N-(2-(5-chloro-2-(1-ethyl-3-methyl-1H-pyrazol-4-ylamino)pyrimidin-4-ylamino)phenyl)methanesulfonamide;
N-(2-(2-(1-ethyl-3-methyl-1H-pyrazol-4-ylamino)-5-fluoropyrimidin-4-ylamino)-5-methoxyphenyl)methanesulfonamide;
N-(2-(5-chloro-2-(1,3-dimethyl-1H-pyrazol-4-ylamino)pyrimidin-4-ylamino)phenyl)methanesulfonamide;
N-(2-(5-fluoro-2-(isoxazol-3-ylamino)pyrimidin-4-ylamino)-5-methoxyphenyl)methanesulfonamide;
N-(2-(2-(1-ethyl-3-methyl-1H-pyrazol-4-ylamino)-5-fluoropyrimidin-4-ylamino)phenyl)methanesulfonamide;
N-(2-(5-chloro-2-(isoxazol-3-ylamino)pyrimidin-4-ylamino)phenyl)methanesulfonamide;
N-(2-(5-chloro-2-(1,5-dimethyl-1H-pyrazol-3-ylamino)pyrimidin-4-ylamino)phenyl)methanesulfonamide;
N-(2-(5-chloro-2-(1-isopropyl-1H-pyrazol-3-ylamino)pyrimidin-4-ylamino)phenyl)methane sulfonamide;
N-(2-(5-bromo-2-(1-methyl-1H-pyrazol-4-ylamino)pyrimidin-4-ylamino)phenyl)methane sulfonamide;
N-(2-(5-chloro-2-(1-ethyl-1H-pyrazol-3-ylamino)pyrimidin-4-ylamino)phenyl)methane sulfonamide;
N-(2-((5-bromo-2-((1-methyl-1H-pyrazol-3-yl)amino)pyrimidin-4-yl)amino)phenyl)methanesulfonamide;
N-(2-((5-chloro-2-(isoxazol-4-ylamino)pyrimidin-4-yl)amino)phenyl)methanesulfonamide;
N-(2-((5-chloro-2-((3-methylisoxazol-5-yl)amino)pyrimidin-4-yl)amino)phenyl)methanesulfonamide;
N-(2-((5-chloro-2-((1-propyl-1H-pyrazol-3-yl)amino)pyrimidin-4-yl)amino)phenyl)methanesulfonamide;
N-(2-((2-((1,5-dimethyl-1H-pyrazol-3-yl)amino)-5-fluoropyrimidin-4-yl)amino)-5-methoxyphenyl)methane sulfonamide;
N-(2-((5-fluoro-2-((1-isopropyl-1H-pyrazol-3-yl)amino)pyrimidin-4-yl)amino)-5-methoxyphenyl)methane sulfonamide;
N-(2-((5-bromo-2-((1-isopropyl-1H-pyrazol-3-yl)amino)pyrimidin-4-yl)amino)phenyl)methanesulfonamide;
N-(2-((5-bromo-2-(isoxazol-3-ylamino)pyrimidin-4-yl)amino)phenyl)methanesulfonamide;
N-(2-((5-bromo-2-((1,5-dimethyl-1H-pyrazol-3-yl)amino)pyrimidin-4-yl)amino)phenyl)methanesulfonamide;
N-(2-((5-chloro-2-((1-(2-methoxyethyl)-1H-pyrazol-4-yl)amino)pyrimidin-4-yl)amino)phenyl)methanesulfonamide
N-(2-((5-chloro-2-((1-methyl-1H-pyrazol-3-yl)amino)pyrimidin-4-yl)amino)phenyl)cyclopropanesulfonamide;
N-(2-((5-chloro-2-((1-isopropyl-1H-pyrazol-3-yl)amino)pyrimidin-4-yl)amino)phenyl)cyclopropanesulfonamide;
N-(2-((5-chloro-2-((1,5-dimethyl-1H-pyrazol-3-yl)amino)pyrimidin-4-yl)amino)phenyl)cyclopropanesulfonamide;
N-(2-((5-chloro-2-((1-methyl-1H-pyrazol-3-yl)amino)pyrimidin-4-yl)amino)phenyl)ethanesulfonamide;
N-(2-((5-chloro-2-((1-methyl-1H-pyrazol-4-yl)amino)pyrimidin-4-yl)amino)phenyl)ethanesulfonamide;
N-(2-((5-chloro-2-((1-methyl-1H-pyrazol-4-yl)amino)pyrimidin-4-yl)amino)phenyl)cyclopropanesulfonamide;
N-(2-((5-chloro-2-((1-ethyl-5-methyl-1H-pyrazol-3-yl)amino)pyrimidin-4-yl)amino)phenyl)methanesulfonamide;
N-(2-((5-chloro-2-((1-(cyclopropylmethyl)-1H-pyrazol-5-yl)amino)pyrimidin-4-yl)amino)-5-methoxyphenyl)methanesulfonamide;
N-(2-((2-((1,5-dimethyl-1H-pyrazol-3-yl)amino)-5-fluoropyrimidin-4-yl)amino)phenyl)ethanesulfonamide;
N-(2-((5-fluoro-2-((1-methyl-1H-pyrazol-4-yl)amino)pyrimidin-4-yl)amino)phenyl)ethanesulfonamide;
N-(2-((5-fluoro-2-((1-methyl-1H-pyrazol-3-yl)amino)pyrimidin-4-yl)amino)phenyl)ethanesulfonamide;
N-(2-((5-fluoro-2-((1-(2-methoxyethyl)-1H-pyrazol-3-yl)amino)pyrimidin-4-yl)amino)phenyl)ethanesulfonamide;
N-(2-((5-chloro-2-((1-(3-cyanopropyl)-1H-pyrazol-4-yl)amino)pyrimidin-4-yl)amino)phenyl)methanesulfonamide;
N-(2-((2-((1-(3-cyanopropyl)-1H-pyrazol-4-yl)amino)-5-fluoropyrimidin-4-yl)amino)phenyl)methanesulfonamide;
N-(2-((5-chloro-2-((1,5-dimethyl-1H-pyrazol-4-yl)amino)pyrimidin-4-yl)amino)-5-methoxyphenyl)methanesulfonamide;
N-(2-((5-chloro-2-((1-isopropyl-1H-pyrazol-5-yl)amino)pyrimidin-4-yl)amino)-5-methoxyphenyl)methanesulfonamide;
N-(2-((5-chloro-2-((3-methyl-1-propyl-1H-pyrazol-5-yl)amino)pyrimidin-4-yl)amino)-5-methoxyphenyl)methanesulfonamide;
N-(2-((5-chloro-2-((1-methyl-1H-pyrazol-3-yl)amino)pyrimidin-4-yl)amino)-6-fluorophenyl)methanesulfonamide;
N-(2-((5-chloro-2-((1-isopropyl-3-methyl-1H-pyrazol-5-yl)amino)pyrimidin-4-yl)amino)-5-methoxyphenyl)methanesulfonamide;
N-(2-((2-((1-(2,2-difluoroethyl)-3-methyl-1H-pyrazol-4-yl)amino)-5-fluoropyrimidin-4-yl)amino)phenyl)methanesulfonamide;
N-(2-((2-((1-(2,2-difluoroethyl)-3-methoxy-1H-pyrazol-4-yl)amino)-5-fluoropyrimidin-4-yl)amino)phenyl)methanesulfonamide;
N-(2-((5-fluoro-2-((3-methoxy-1-(2,2,2-trifluoroethyl)-1H-pyrazol-4-yl)amino)pyrimidin-4-yl)amino)phenyl)methanesulfonamide;
N-(2-((5-fluoro-2-((5-methyl-1-(2,2,2-trifluoroethyl)-1H-pyrazol-3-yl)amino)pyrimidin-4-yl)amino)phenyl)methanesulfonamide;
N-(2-((5-chloro-2-((1-propyl-1H-pyrazol-5-yl)amino)pyrimidin-4-yl)amino)-5-methoxyphenyl)methanesulfonamide;
N-(2-((5-chloro-2-((1-ethyl-5-methyl-1H-pyrazol-4-yl)amino)pyrimidin-4-yl)amino)-5-methoxyphenyl)methanesulfonamide;
N-(2-((5-chloro-2-((1,5-dimethyl-1H-pyrazol-3-yl)amino)pyrimidin-4-yl)amino)-6-fluorophenyl)methanesulfonamide;
N-(2-((5-chloro-2-((1-propyl-1H-pyrazol-3-yl)amino)pyrimidin-4-yl)amino)-6-fluorophenyl)methanesulfonamide;
N-(2-((5-chloro-2-((1-(2,2-difluoroethyl)-3-methoxy-1H-pyrazol-4-yl)amino)pyrimidin-4-yl)amino)phenyl)methanesulfonamide;
N-(2-((5-chloro-2-((1-(2,2-difluoroethyl)-3-methyl-1H-pyrazol-4-yl)amino)pyrimidin-4-yl)amino)phenyl)methanesulfonamide;
N-(2-((5-chloro-2-((3-methoxy-1-(2,2,2-trifluoroethyl)-1H-pyrazol-4-yl)amino)pyrimidin-4-yl)amino)phenyl)methanesulfonamide;
N-(2-((5-chloro-2-((5-methyl-1-(2,2,2-trifluoro ethyl)-1H-pyrazol-3-yl)amino)pyrimidin-4-yl)amino)phenyl)methanesulfonamide;
N-(2-((5-chloro-2-((1-(2,2-difluoroethyl)-1H-pyrazol-4-yl)amino)pyrimidin-4-yl)amino)phenyl)methanesulfonamide;
N-(2-((2-((1-(2,2-difluoro ethyl)-1H-pyrazol-4-yl)amino)-5-fluoropyrimidin-4-yl)amino)phenyl)methanesulfonamide;
N-(2-((2-((1-butyl-3-methyl-1H-pyrazol-5-yl)amino)-5-chloropyrimidin-4-yl)amino)-5-methoxyphenyl)methanesulfonamide;
N-(2-((2-((1-butyl-1H-pyrazol-5-yl)amino)-5-chloropyrimidin-4-yl)amino)-5-methoxyphenyl)methanesulfonamide;
N-(2-((5-chloro-2-((1-(1-cyclopropylethyl)-3-methyl-1H-pyrazol-5-yl)amino)pyrimidin-4-yl)amino)-5-methoxyphenyl)methanesulfonamide;
N-(2-((5-chloro-2-((1-methyl-1H-pyrazol-4-yl)amino)pyrimidin-4-yl)amino)-6-fluorophenyl)methanesulfonamide;
N-(2-((5-chloro-2-((1-ethyl-1H-pyrazol-3-yl)amino)pyrimidin-4-yl)amino)-6-fluorophenyl)methanesulfonamide;
N-(2-((5-chloro-2-((1-ethyl-5-methyl-1H-pyrazol-3-yl)amino)pyrimidin-4-yl)amino)-6-fluorophenyl)methanesulfonamide;
3-((5-chloro-4-((2-(methylsulfonamido)phenyl)amino)pyrimidin-2-yl)amino)-1-methyl-1 H-pyrazole-5-carboxylic acid;
N-(2-((5-chloro-2-((1-(2-methoxyethyl)-1H-pyrazol-4-yl)amino)pyrimidin-4-yl)amino)-6-fluorophenyl)methanesulfonamide;
N-(2-((5-chloro-2-((1-(2-hydroxyethyl)-3-methyl-1H-pyrazol-5-yl)amino)pyrimidin-4-yl)amino)-5-methoxyphenyl)methanesulfonamide;
N-(2-((5-chloro-2-((1-isobutyl-3-methyl-1H-pyrazol-5-yl)amino)pyrimidin-4-yl)amino)-5-methoxyphenyl)methanesulfonamide;
N-(2-((5-chloro-2-((1-(2-methoxyethyl)-3-methyl-1H-pyrazol-5-yl)amino)pyrimidin-4-yl)amino)-5-methoxyphenyl)methanesulfonamide;
3-((5-chloro-4-((2-(methylsulfonamido)phenyl)amino)pyrimidin-2-yl)amino)-N,1-dimethyl-1H-pyrazole-5-carboxamide
N-(2-((5-chloro-2-((1-isobutyl-1H-pyrazol-5-yl)amino)pyrimidin-4-yl)amino)-5-methoxyphenyl)methanesulfonamide;
N-(2-fluoro-6-((5-fluoro-2-((1-methyl-1H-pyrazol-4-yl)amino)pyrimidin-4-yl)amino)phenyl)methanesulfonamide;
3-((5-chloro-4-((2-(methylsulfonamido)phenyl)amino)pyrimidin-2-yl)amino)-N-ethyl-1-methyl-1H-pyrazole-5-carboxamide; and
3-((5-chloro-4-((2-(methylsulfonamido)phenyl)amino)pyrimidin-2-yl)amino)-N-(2-hydroxyethyl)-1-methyl-1H-pyrazole-5-carboxamide.

12. A compound of claim 1, a pharmaceutically acceptable salt or tautomer thereof.

13. A pharmaceutical composition comprising a compound or a pharmaceutically acceptable salt thereof of claim 1 together with a pharmaceutically acceptable carrier, optionally in combination with one or more other pharmaceutical compositions.

14. (canceled)

15. (canceled)

16. (canceled)

17. A method for treating, controlling, delaying or preventing in a mammalian patient in need of the treatment of one or more conditions selected from the group consisting of diseases and disorders associated with ZAP-70, Syk or JAK3 wherein the method comprises the administration to said patient a therapeutically effective amount of a compound of claim 1 or a pharmaceutically acceptable salt thereof.

18. A method for treating, controlling, delaying or preventing in a mammalian patient in need of the treatment of one or more conditions selected from the group consisting of immunological, inflammatory, autoimmune, allergic disorders, and immunologically-mediated diseases, wherein the method comprises the administration to said patient a therapeutically effective amount of a compound of claim 1 or a pharmaceutically acceptable salt thereof.

19. Method for the preparation of a compound of formula (I) of claim 1, comprising the steps of
(a) reacting a compound of formula (II) 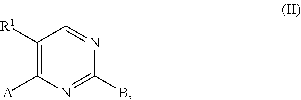
wherein R¹ has the meaning as in claim 1 and A, B are suitable leaving groups with one of the compounds of formula (III) or (IV) 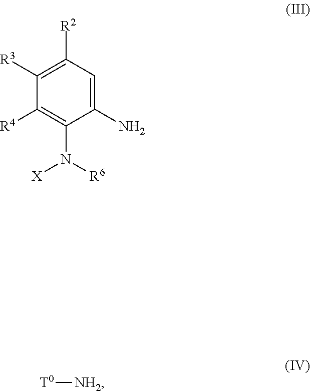
wherein X is S(O)₂R⁵; or H and wherein R², R³, R⁴, R⁵, R⁶, T⁰ have the meaning as indicated above and;
(b) reacting the resulting product from step (a) with the other compound of formula (III) or (IV) to yield a compound of formula (I) when X is S(O)₂R⁵; or
(c) reacting the resulting product of step (b) when X is H with a compound of formula R⁵S(O)₂C1 to yield a compound of formula (I).
